(19) United States
(12) Patent Application Publication
(10) Pub. No.: US 2004/0048241 A1
(43) Pub. Date: Mar. 11, 2004

(54) Methods for attaching molecules

(76) Inventors: Beverly Annette Freeman, Albany, CA (US); Sobha Pisharody, Castro Valley, CA (US)

Correspondence Address: JONES DAY, 222 EAST 41ST STREET, NEW YORK, NY, 10017, US

(21) Appl. No.: 10/330,445

(22) Filed: Dec. 26, 2002

Related U.S. Application Data
(63) Continuation-in-part of application No. 09/970,087, filed on Oct. 2, 2001.
(60) Provisional application No. 60/297,583, filed on Jun. 11, 2001.
(60) Provisional application No. 60/372,933, filed on Apr. 17, 2002.

(51) Int. Cl. C12Q 1/70; C12Q 1/68; G01N 33/53; G01F 1/64; G01N 27/26

(52) U.S. Cl. 435/5; 435/6; 435/7.1; 205/777.5

(57) ABSTRACT

A method of electrically coupling an electrode pair in a plurality of electrode pairs in a biosensor with a macromolecule. The electrode pair comprises a first electrode and a second electrode. A first portion of the macromolecule is derivatized with a first reactive group and a second portion of the macromolecule is derivatized with a second reactive group. The first reactive group is masked with a first electrolabile masking group and the second reactive group is masked with a second electrolabile masking group. A first voltage is applied at the first electrode in the electrode pair under conditions that are sufficient to unmask the first reactive group. The unmasked first reactive group binds to the first electrode thereby linking the macromolecule to the first electrode. A second voltage is applied at the second electrode in the electrode pair under conditions that are sufficient to unmask the second reactive group. The unmasked second reactive group binds to the second electrode thereby electrically coupling the electrode pair in the biosensor with the macromolecule.

### 1. CROSS-REFERENCE TO RELATED APPLICATIONS

This application is a continuation-in-part of U.S. patent application Ser. No. 09/970,087, filed Oct. 2, 2001, which claims the benefit of U.S. Provisional Patent Application No. 60/297,583, filed on Jun. 11, 2001, each of which is incorporated herein be reference in their entireties. Furthermore, this application claims priority, under 35 U.S.C. § 119(e), of U.S. Provisional Patent Application No. 60,372,933 filed on Apr. 17, 2002 which is incorporated herein, by reference, in its entirety.

### 2. FIELD OF THE INVENTION

The present invention relates to the association of molecules with electrodes in an array of electrodes. In particular, different molecules can be selectively associated with different electrodes in an array of electrodes.

### 3. BACKGROUND OF THE INVENTION

Biosensors are devices that can detect and/or quantify analytes using known interactions between a targeted analyte and a binding agent that is typically a biological macromolecule, such as an enzyme, receptor, nucleic acid, protein, lectin, or antibody. Biosensors have applications in virtually all areas of human endeavor. For example, biosensors have utility in fields as diverse as blood glucose monitoring for diabetics, the recognition of poisonous gas and/or explosives, the detection of chemicals commonly associated with spoiled or contaminated food, genetic screening, environmental testing, and the like. Thus, the term “biosensor” refers to a sensor that uses a biological macromolecule (e.g. nucleic acid, carbohydrate, protein, antibody, etc.) to specifically recognize/bind to a target analyte. The term “molecular sensing apparatus” is used interchangeably with the term “biosensor”.

Biosensors are commonly categorized according to two features, namely, the type of macromolecule utilized in the device and the means for detecting the contact between the binding agent and the targeted analyte. Major classes of biosensors include enzyme (or catalytic) biosensors, immunosensors and DNA biosensors.

Enzyme (or catalytic) biosensors typically utilize one or more enzymes as the macromolecule and take advantage of the complimentary shape of the selected enzyme and the targeted analyte. Enzymes are proteins that perform most of the catalytic work in biological systems and are known for highly specific catalysis. The shape and reactivity of a given enzyme limits its catalytic activity to a very small number of possible substrates. Enzyme biosensors rely on the specific chemical changes related to the enzyme/analyte interaction as the means for recognizing contact with the targeted analyte. For example, upon interaction with an analyte, an enzyme biosensor may generate electrons, a colored chromophore or a change in pH as the result of the relevant enzymatic reaction. Alternatively, upon interaction with an analyte, an enzyme biosensor may cause a change in a fluorescent or chemiluminescent signal that can be recorded by an appropriate detection system.

Immunosensors utilize antibodies as binding agents. Antibodies are protein molecules that generally do not perform catalytic reactions, but specifically bind to particular “target” molecules (antigens). Antibodies are quite specific in their interactions and, unlike most enzymes, they are capable of recognizing and selectively binding to very large bodies such as single cells. Thus, in addition to detection of small analytes, antibody-based biosensors allow for the identification of certain pathogens such as dangerous bacterial strains.

DNA biosensors typically utilize the complementary nature of DNA double-strands. They are designed for the specific detection of particular nucleic acids. A DNA biosensor sensor generally uses a single-stranded DNA as the binding agent. The nucleic acid material in a given test sample is placed into contact with the binding agent under conditions where the biosensor DNA and the target nucleic acid analyte can form a hybrid duplex. If a nucleic acid in the test sample is complementary to a nucleic acid used in the biosensor, the two interact (e.g., the two bind to each other). The interaction can be monitored by various means such as a change in mass at the sensor surface or the presence of a fluorescent or radioactive signal. In alternative arrangements, the target nucleic acid(s) are bound to the sensor and contacted with labeled probes to allow for identification of the sequence(s) of interest.

When a single-stranded DNA binds to a complementary single-stranded DNA or RNA, the charge conducting characteristics of the DNA change. Charge transfer and transport in DNA is a function of many different phenomena, including the redox potential of the bases in the DNA, base-stacking characteristics, structural distortion, as well as the sequence of the DNA. See, for example, Cai et al., 2000, Applied Physics Letters 77, pp. 3105-3106; and Giese et al., 2001, Nature 412, pp. 318-320. Further, the studies of Fink & Schönenberger, Hjort & Stafström, and Kasumov et al. indicate that at least some DNA sequences are molecular conductors. See Fink & Schönenberger, 1999, Nature 398, pp. 407-410; Hjort & Stafström, 2001, Physical Review Letters 87, 228101-1228101-4; Kasumov et al., 2001, Science 291, pp. 280-282.

While biosensors have potential and while biosensors have been described in patents and in the literature, actual commercial use of biosensors remains limited. Features of biosensors that have limited their commercial acceptance include the difficulty in selectively binding probes to each electrode pair in the biosensors. For example, in the case where a plurality of binding agents are required, the time required to selectively spatially bind the different binding agents with the biosensor is unduly long. Thus, the ability to rapidly and selectively associate molecules with selected surfaces has importance in the fabrication and commercial utility of biosensors.

Thus, given the above background, what is needed in the art are improved methods for selectively binding agents to electrode pairs in biosensors.

### 4. SUMMARY OF THE INVENTION

This invention pertains to methods of making sensors (biosensors) that are useful for detecting a wide range of macromolecules as well as macromolecule binding events. The biosensors of the present invention include a plurality of electrode pairs. In the present invention, a binding agent includes a reactive group that allows the binding agent to react with an electrode in an electrode pair of the biosensor and thereby bind to the electrode. However, the reactive group is initially masked with an electrolabile masking group. This electrolabile group prevents the binding agent from binding to the biosensor. A voltage is applied at a specific electrode within the biosensor. The applied voltage causes the electrolabile masking group to undergo a chemical reaction such that the group no longer masks the reactive group on the binding agent. Therefore, binding agent in solution near the electrode in which voltage has been applied selectively binds to the activated electrode. In this way, electrodes in a biosensor can be selectively populated with specific binding agents.

4.1 Electrode Specific Biological Macromolecule Binding using Applied Voltages

One aspect of the present invention provides a method of electrically coupling an electrode pair in a plurality of electrode pairs in a biosensor with a macromolecule. The electrode pair comprises a first electrode and a second electrode. A first portion of the macromolecule is derivatized with a first reactive group and a second portion of the macromolecule is derivatized with a second reactive group. The first reactive group is masked with a first electrolabile masking group and the second reactive group is masked with a second electrolabile masking group. The method comprises applying a first voltage at the first electrode in the electrode pair under conditions that are sufficient to unmask the first reactive group. The unmasked first reactive group binds to the first electrode thereby linking the macromolecule to the first electrode. A second voltage is applied at the second electrode in the electrode pair under conditions that are sufficient to unmask the second reactive group. The unmasked second reactive group binds to the second electrode thereby electrically coupling the electrode pair in the biosensor with the macromolecule.

In some embodiments, the first voltage and the second voltage are different. In some embodiments, the first electrode and the second electrode are separated by a distance that is between 10 Angstroms and 10,000 Angstroms, between 30 Angstroms and 500 Angstroms, or between 50 Angstroms and 200 Angstroms. In some embodiments, the first electrode and the second electrode have a resistivity of less than 10⁻⁴ ohm meters.

In some embodiments, the first electrode and the second electrode are each made from the same or different material that are each independently selected from the group consisting of silicon, dense silicon carbide, boron carbide, Fe₃O₄, germanium, silicone germanium, silicon carbide, polysilicon, tungsten carbide, titanium carbide, indium phosphide, gallium nitride, gallium phosphide, aluminum phosphide, aluminum arsenide, mercury cadmium telluride, tellurium, selenium, ZnS, ZnO, ZnSe, CdS, ZnTe, GaSe, CdSe, CdTe, GaAs, InP, GaSb, InAs, Te, PbS, InSb, InSb, PbTe, PbSe, tungsten disulfide.

In some embodiments, the first electrode and the second electrode are made of a metal. In some embodiments, the first electrode and the second electrode are each made from the same or different material that are each independently selected from the group consisting of ruthenium, cobalt, rhodium, rubidium, lithium, sodium potassium, vanadium, cesium, chromium, molybdenum, silicon, germanium, aluminum, iridium, nickel, palladium, platinum, iron, copper, titanium, tungsten, silver, gold, zinc, cadmium, indium tin oxide, carbon, and carbon nanotube and an alloy thereof.

In some embodiments of the present invention, the macromolecule comprises a nucleic acid, a protein, a polypeptide, a peptide, an antibody, a carbohydrate, a polysaccharide, a lipid, a fatty acid or a sugar. In some embodiments, the first reactive group and the second reactive group are the same or different material and are each independently selected from the group consisting of a sulfate, a sulfonate, a sulfonyl moiety, a thiol, a thioether, a sulfur-containing moiety, a chalcogen-containing moiety, an amine, a carbonyl-containing moiety, a carboxylic acid, an aldehyde, a ketone, a phosphate, a phosphonate, a phosphorothioate, a pnictogen moiety, a silane, a silicon-containing moiety, an alkene, an alkyne, a hydroxyl group, and a halogen.

In some embodiments, the first reactive group and the second reactive group are each a thiol and the first electrolabile masking group and the second electrolabile masking group are different and are each independently selected from the group consisting of an S-2,2,2-trichloroethoxycarbonyl derivative, an S-benzyloxycarbonyl derivative, an S-benzyl thioether derivative, an S-triphenylmethyl thioether derivative, an S-2,4,6-trimethoxybenzyl thioether derivative, and an S-2-picolyl N-oxide thioether derivative.

In some embodiments, the method further comprises (i) testing for background conductance between the first electrode and the second electrode, (ii) exposing the electrode pair to a solution that potentially comprises an analyte for a period of time, (iii) drying the electrode pair, and (iv) measuring a current through the electrode pair. In some embodiments, the period of time is less than one minute, less than 30 minutes, or between one minute and one hour. In some embodiments, the drying step comprises blowing nitrogen or argon gas on the electrode pair. In some embodiments, the measuring step comprises quantifying a current across the electrode pair when a voltage of between ±3 volts, between ±5 volts, or between ±7 volts is applied across the electrode pair.

In some embodiments of the present invention, the macromolecule is a single stranded nucleic acid and the analyte is a single stranded nucleic acid that has sufficient complementarity to the macromolecule to bind to the macromolecule in the exposing step. In some embodiments of the present invention, the macromolecule is a single stranded nucleic acid and the analyte is a single stranded nucleic acid that has sufficient complementarity to the macromolecule to bind to the macromolecule under conditions of high stringency, intermediate stringency, or low stringency.

In some embodiments, the analyte comprises a whole cell, a subcellular particle, a virus, a prion, a viroid, a nucleic acid, a protein, an antigen, a lipoprotein, a lipopolysaccharide, a lipid, a glycoproteins, a carbohydrate moiety, a cellulose derivative, an antibody, a fragment of an antibody, a peptide, a hormone, a pharmacological agent, a cellular component, an organic compound, a non-biological polymer, a synthetic organic molecule, an organo-metallic compound, or an inorganic molecule.

In some embodiments of the present invention, the first and second applying steps are repeated with a different electrode pair in the plurality of electrode pairs using a different macromolecule. In such embodiments, the different electrode pair comprises a first electrode and a second electrode, a first portion of the different macromolecule is derivatized with a first reactive group and a second portion of the macromolecule is derivatized with a second reactive group. Further, the first reactive group is masked with a first electrolabile masking group and the second reactive group is masked with a second electrolabile masking group.

In some embodiments of the present invention, the first applying step is repeated with a different electrode pair in the plurality of electrode pairs using a different macromolecule prior to the second applying step. In such embodiments, the different electrode pair comprises a first electrode and a second electrode and a first portion of the different macromolecule is derivatized with a first reactive group and a second portion of the macromolecule is derivatized with a second reactive group. Furthermore, the first reactive group is masked with a first electrolabile masking group and the second reactive group is masked with a second electrolabile masking group.

4.2 Electrode Specific Biological Macromolecule Binding using an Intercalator

Another aspect of the present invention provides a method of electrically coupling an electrode pair in a plurality of electrode pairs in a biosensor with a macromolecule. In this aspect of the invention, (i) the electrode pair comprises a first electrode and a second electrode, (ii) an intercalator is covalently linked to the second electrode, and (iii) a portion of the macromolecule is derivatized with a reactive group that is masked with an electrolabile masking group. In the method, a voltage is applied at the first electrode in the electrode pair under conditions that are sufficient to unmask the reactive group. The unmasked reactive group binds to the first electrode thereby linking the macromolecule to the first electrode. Further, the electrode pair is exposed to a solution that potentially comprises an analyte for a period of time. When the analyte binds to the macromolecule to form a complex comprising the macromolecule and the analyte, the intercalator binds to the complex thereby electrically connecting the electrode pair.

In some embodiments of the present invention, the reactive group is selected from the group consisting of a sulfate, a sulfonate, a sulfonyl moiety, a thiol, a thioether, a sulfur-containing moiety, a chalcogen-containing moiety, an amine, a carbonyl-containing moiety, a carboxylic acid, an aldehyde, a ketone, a phosphate, a phosphonate, a phosphorothioate, a pnictogen moiety, a silane, a silicon-containing moiety, an alkene, an alkyne, a hydroxyl group, and a halogen. In some embodiments, the macromolecule is a single stranded nucleic acid and the analyte is a single stranded nucleic acid that has sufficient complementarity to the macromolecule to bind to the macromolecule in the exposing step.

In some embodiments, the intercalator comprises ethidium, an ethidium derivative, an ethidium complex, acridine, an acridine derivative or an acridine complex. As used herein, an ethidium derivative is a covalent modification of ethidium whereas an ethidium complex is a complex in which an ethidium or a ethidium derivative is noncovalently complexed with another chemical entity.

In some embodiments, the intercalator comprises acridine orange, acridine yellow, 9-aminoacridine, hydrochloride hydrate, 2-aminoacridone, 9,9′-biacridyl, 9-chloroacridine, 6,9-dichloro-2-methoxyacridine, n-(1-leucyl)-2-aminoacridone, 10-octadecyl acridine orange, rivanol, doxorubicin, daunorubicin, actinomycin D, 7-amino Actinomycin D, ellipticine, coralyne, propidium, TAS103, berberine, distamycin, berenil, 7H-methylbenzo[e]pyrido[4,3-b]indole, meso-tetrakis(N-methyl-4pyridyl)porphine, N-methyl mesoporphyrin, diamidino-2phenylindole, 1 -pyrenemethylamine hydrochloride, netropsin, hoeschst 33342, hoeschst 33258, hoeschst 8208, naphthalene diimide, or ethidium bromide.

In some embodiments, the method further comprises drying the electrode pair and measuring a current through the electrode pair. In some embodiments, this drying comprises blowing nitrogen or argon gas on the electrode pair. In some embodiments, the measuring step (d) comprises quantifying a current across the electrode pair when a voltage of between ±3 volts, between ±5 volts, or between ±7 volts is applied across the electrode pair.

In some embodiments, the applying step is repeated, before the exposing step, with a different electrode pair in the plurality of electrode pairs using a different macromolecule. In such embodiments, (i) the different electrode pair comprises a first electrode and a second electrode, and (ii) a portion of the different macromolecule is derivatized with a reactive group that is masked with a electrolabile masking group.

In some embodiments, the applying step and the exposing step are repeated with a different electrode pair in the plurality of electrode pairs using a different macromolecule. Furthermore, the different electrode pair comprises a first electrode and a second electrode, and (ii) a portion of the different macromolecule is derivatized with a reactive group that is masked with a electrolabile masking group.

4.3 Electrode Specific Biological Macromolecule Binding using an Electrolabile Masking Group and a Photosensitive or Chemically Sensitive Masking Group

Still another aspect of the present invention provides a method of electrically coupling an electrode pair in a plurality of electrode pairs in a biosensor with a macromolecule. In this aspect of the invention, the electrode pair comprises a first electrode and a second electrode. A first portion of the macromolecule is derivatized with a first reactive group and a second portion of the macromolecule is derivatized with a second reactive group. Further, the first reactive group is masked with an electrolabile masking group and the second reactive group is masked with a photosensitive or chemically sensitive masking group. In the method, a voltage is applied at the first electrode in the electrode pair under conditions that are sufficient to unmask the first reactive group. The unmasked first reactive group binds to the first electrode thereby linking the macromolecule to the first electrode. The electrode pair is exposed to a light source or a chemical thereby unmasking the second reactive group. The unmasked second reactive group binds to the second electrode thereby electrically coupling the electrode pair in the biosensor with the macromolecule.

In some embodiments, the second reactive group is masked with a photosensitive masking group and the light source is ultraviolet or laser light. In some embodiments, the second reactive group is masked with a photosensitive masking group having the formula:

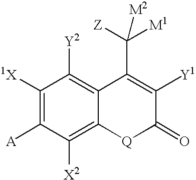

wherein,

A is —OH, substituted or unsubstituted alkoxy, —OC(O)CH₃, —NH₂, or —NHCH₃;

each of X¹ and X², independently, is H, Cl, Br, or I, at least one of X¹ and X² being Cl, Br, or I;

Q is —O—, —NH—, or —NCH₃—;

Y¹ is —H, —Cl, —Br, —I, —C(O)OH, —NO₂, —C(O)NHR¹, —CN, —C(O)H, —C(O)CH₃, benzoxazol-2-yl, benzothiazol-2-yl, or benzimidazol-2-yl;

Y² is —H, —C(O)OH, or —SO₃H; M¹ is —H, —CH₃, —NR²R³, —C(O)NR² R³, or —COOH;

Z is the second reactive group;

M² is —H, or Z and M² together are ═N₂, ═O, or ═NNHR¹; and

each of R¹, R², and R³, independently, is a substituted or unsubstituted moiety selected from the group consisting of a C₁₋₂₀ alkyl, a C₂₋₂₀ alkenyl, a C₂₋₂₀ alkynyl, a C₁₋₂₀ alkoxy, a C₁₋₂₀ thioalkoxy, a C₁₋₂₀ alkylsulfonyl, a C₄₋₁₆ arylsulfonyl, a C₂₋₂₀ heteroalkyl, a C₂₋₂₀ heteroalkenyl, a C₃₋₈ cycloalkyl, a C₃₋₈ cycloalkenyl, a C₄₋₁₆ aryl, a C₄₋₁₆ heteroaryl, and a C₂₋₃₀ heterocyclyl.

In some embodiments of the present invention, the second reactive group and the chemically sensitive masking group together form a moiety selected from the group consisting of an s-alkyl thioether having the formula CₙH₂ₙ₊₁SR, an s-benzyl thioether having the formula RSCh₂Ph, and an s-diphenylmethyl thioether having the formula RSCH(C₆H₅)₂, wherein R is the macromolecule; and the exposing step cleaves the moiety thereby unmasking the second reactive group.

In some embodiments in accordance with this aspect of the invention, the method further comprises (i) testing for background conductance between the first electrode and the second electrode, (ii) exposing the electrode pair to a solution that potentially comprises an analyte for a period of time, (iii) drying the electrode pair, and (iv) and measuring a current through the electrode pair.

In some embodiments, the applying step and the exposing step are repeated with a different electrode pair in the plurality of electrode pairs using a different macromolecule. In such embodiments, (i) the different electrode pair comprises a first electrode and a second electrode, (ii) a first portion of the different macromolecule is derivatized with a first reactive group and a second portion of the macromolecule is derivatized with a second reactive group, and (iii) the first reactive group is masked with an electrolabile masking group and the second reactive group is masked with a photosensitive or chemically sensitive masking group.

In some embodiments of the present invention, the applying step is repeated with a different electrode pair in the plurality of electrode pairs using a different macromolecule prior to the exposing step. In such embodiments, the different electrode pair comprises a first electrode and a second electrode. A first portion of the different macromolecule is derivatized with a first reactive group and a second portion of the macromolecule is derivatized with the second reactive group. Further, the first reactive group is masked with an electrolabile masking group and the second reactive group is masked with the photosensitive or chemically sensitive masking group.

### 5. BRIEF DESCRIPTION OF THE DRAWINGS

FIGS. 1A-1E illustrate a method of attaching a biological macromolecule to a biosensor in accordance with one embodiment of the present invention.

FIGS. 2A-2E illustrate a method of attaching a biological macromolecule to a biosensor in accordance with another embodiment of the present invention.

FIGS. 3A-3F illustrate a method of attaching a biological macromolecule to a biosensor in accordance with yet another embodiment of the present invention.

FIG. 4 illustrates a biosensor configuration in accordance with one embodiment of the present invention.

FIG. 5 illustrates a biosensor configuration in accordance with one embodiment of the present invention.

FIG. 6 illustrates a biosensor configuration in accordance with another embodiment of the present invention.

FIGS. 7A-7F illustrate biosensor configurations in accordance with various embodiments of the present invention.

FIG. 8 illustrates an array of devices in a biosensor in accordance with one embodiment of the present invention.

FIGS. 9A-9B illustrate a method of packaging biosensors in accordance with one embodiment of the present invention.

FIG. 10 illustrates a packaged biosensor in accordance with one embodiment of the present invention.

Like reference numerals refer to corresponding parts throughout the several views of the drawings.

### 6. DETAILED DESCRIPTION

This invention pertains to methods of using sensors (biosensors) for the detection of a wide range of macromolecules as well as macromolecule binding events. The biosensors of the present invention include a plurality of electrode pairs. In the present invention, a binding agent includes a reactive group that allows the binding agent to react with an electrode in an electrode pair of the biosensor and thereby bind to the electrode. In some embodiments, the reactive group is initially masked with an electrolabile masking group. This electrolabile group prevents the binding agent from binding to the biosensor. A voltage is applied at a specific electrode or group of electrodes within the biosensor. The applied voltage causes the electrolabile masking group on the binding agent to undergo a chemical reaction such that the masking group no longer masks the reactive group on the binding agent. Therefore, binding agent in solution near the electrode in which voltage has been applied selectively binds to the activated electrode. In this way, electrodes in a biosensor can be selectively populated with specific binding agents. In typical embodiments, the binding agent is a macromolecule 120. Representative macromolecules 120 in accordance with the present invention are found in Section 6.13, below.

The present invention provides several specific embodiments that are advantageous in particular situations. These specific embodiments are described in the following sections.

6.1 Electrode Specific Biological Macromolecule Binding using Applied Voltages

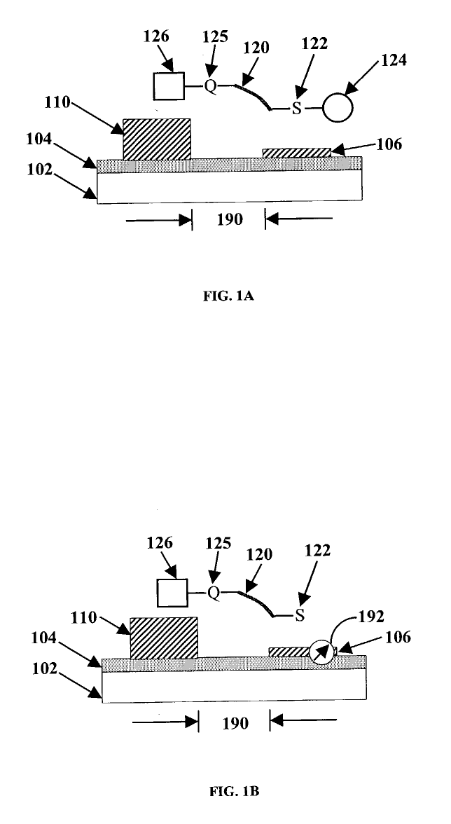

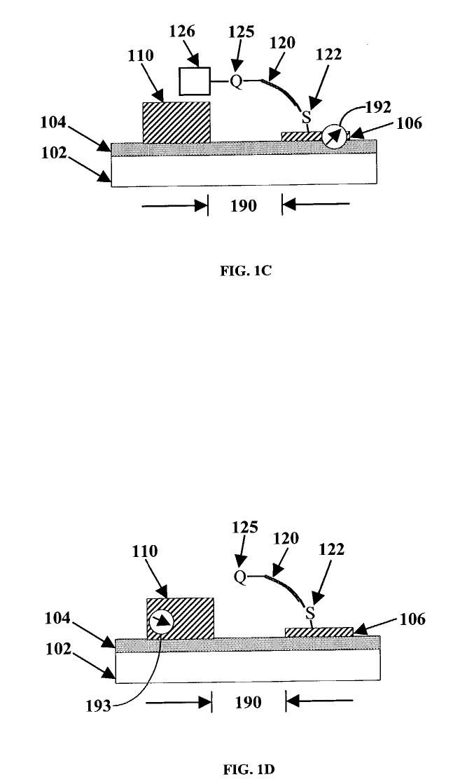

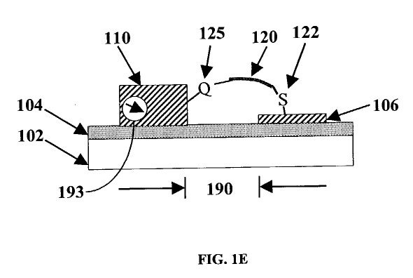

Some embodiments of the present invention make use of applied voltages to bind binding agents (e.g., macromolecules 120) to specific electrodes in an array of electrodes in a biosensor. FIGS. 1A through 1E illustrate one such method in accordance with the present invention. In particular, FIG. 1A illustrates a biosensor that includes a substrate 102 and an optional insulator 104. Representative materials used to make substrate 102 are found in Section 6.9, below. Representative materials used to make optional insulator 104 are found in Section 6.11, below.

The biosensor illustrated in FIG. 1A includes a first electrically conducting material 106 (e.g., a first electrode) and a second electrically conducting material 110 (e.g., a second electrode). Representative materials used to make electrically conducting materials 106 and 110 are found in Section 6.10, below. In embodiments where optional insulator 104 is not used, electrically conducting materials 106 and 110 overlay substrate 102. In some embodiments in accordance with FIG. 1A, material 106 and material 110 are separated by a distance 190 that is between 10 Angstroms and 10,000 Angstroms, between 20 Angstroms and 1,000 Angstroms, between 30 Angstroms and 500 Angstroms, between 40 Angstroms and 300 Angstroms, or between 50 Angstroms and 200 Angstroms. In some embodiments, a distance that is less than 100 Angstroms separates material 106 and material 110. In some embodiments, the distance between materials 106 and 110 is between 10 Angstroms and 100 Angstroms.

FIG. 1A also shows a macromolecule 120 (e.g., binding agent) that has been derivatized with two reactive groups, S and Q. Representative macromolecules 120 are found in Section 6.13, below. In FIG. 1A, reactive groups 122 and 125 are respectively masked by electrolabile masking groups 124 and 126. Representative reactive groups 122 and 125 and representative electrolabile masking groups 124 and 126 are found in Section 6.17, below.

In one example in accordance with the present invention, reactive group 122 and electrolabile masking group 124 together form a thiocarbonate. For example, in some embodiments of the present invention, reactive group 122 is a thiol and electrolabile masking group 124 is an S-2,2,2-trichloroethoxycarbonyl derivative that can be removed at −1.5 volts in the presence of LiClO₄/CH₃OH to reveal the thiol group 122. The reactive thiol group 122 can, in turn, form a covalent bond with an electrically conducting material (e.g., material 106 and/or 110), such as gold. In this example, reactive group 125 and electrolabile masking group 126 also form a thiocarbonate. Reactive group 125 is a thiol and masking group 126 is an s-benzyloxycarbonyl derivative. The s-benzyloxycarbonyl (Cbz) derivative can be removed at −2.6 volts in DMF and tetrabutyl ammonium chloride to reveal a reactive sulfur group. The reactive sulfur group 125 can bind with an electrically conducting material such as gold.

In FIG. 1B, the method continues with the application of a voltage at electrically conducting material 106. The voltage 192 applied in this step is sufficient to unmask reactive group 122 but is not sufficient to unmask group 125. Thus, electrolabile masking group 124 is stripped from the macromolecules 120 that are in the vicinity of electrically conducting material 106 but electrolabile masking group 126 remains attached to such macromolecules. The stripping of electrolabile masking group 124 from the macromolecules 120 that are in the vicinity of electrically conducting material 106 exposes reactive group 122 on such molecules. Exposed reactive groups 122 react with electrically conducting material 106 thereby binding the macromolecules 120 in the vicinity of material 106 to material 106 as illustrated in FIG. 1C. In one example reactive group 122 and electrolabile masking group 124 together form a thiocarbonate. For example, in some embodiments of the present invention, reactive group 122 is a thiol and electrolabile masking group 124 is an S-2,2,2-trichloroethoxycarbonyl derivative that can be removed at −1.5 volts in the presence of LiClO₄/CH₃O H to reveal the thiol group 122. The reactive thiol group 122 can, in turn, form a covalent bond with electrically conducting material 106.

In FIG. 1D, voltage 193 is applied. In some embodiments, voltage 193 (FIG. 1C) is different from voltage 192 (FIG. 1D). Voltage 193 is sufficient to strip electrolabile masking group 126 from macromolecules 120 that are bound to electrically conducting material 106, thereby exposing reactive group 125. Exposed reactive group 125 then interacts with electrically conducting material 110 as illustrated in FIG. 1E. In some embodiments, reactive group 125 is a thiol and electrolabile masking group 126 is a s-benzyloxycarbonyl derivative that can be cleaved at a voltage 193 of −2.6 volts in the presence of DMF and tetrabutyl ammonium chloride to unmask the reactive thiol 125. The unmasked reactive thiol 125 then binds to electrically conducting material 110.

Although specific examples of electrolabile masking groups 124 and 126, reactive groups 122 and 125, and voltages 192 and 193 are described in this section, the present invention is not limited to such examples. Indeed, any set of masking groups 124 and 126 that are labile at different voltages may be used in the present invention. Representative electrolabile masking groups 124 and 126 and reactive groups 122 and 125 that may be used in this embodiment of the present invention are disclosed in Section 6.17, below.

The biosensor configuration illustrated in FIG. 1E has a wide range of uses. For example, in some embodiments, macromolecule 120 is a single stranded nucleic acid. As a result of the methods disclosed in this section, a first portion of macromolecule 120 is attached to a first electrode (electrically conducting material 106) and a second portion of macromolecule 120 is attached to a second electrode (electrically conducting material 110). The configuration shown in FIG. 1E can therefore be used as a molecular binding event sensor.

In one example, the method of the present invention described above and illustrated in FIGS. 1A-1E is used to attach a macromolecule 120 to the biosensor. Next, electrically conducting materials 106 and 110 are connected to a voltage source and tested for non-conductance, or a background conductance, prior to exposing macromolecule 120 to an analyte. Background conductance is measured, using, for example, a high-speed potentiostat/galvanostat (e.g. Perkin-Elmer, Model 283). Then, the bound macromolecule 120 is exposed to a solution that may comprise an analyte. In some embodiments, this exposure period has a duration of less than one minute, less than five minutes, less than 15 minutes, less than 30 minutes, less than an hour, less than four hours, between one minute and one hour, between 1 hour and 15 hours, or less than one day. In some embodiments, this exposure period is between one second and one minute, between one minute and five minutes, between five minutes and fifteen minutes, between fifteen minutes and 30 minutes, between 30 minutes and one hour, or more than one hour. In one example, macromolecule 120 is a single stranded nucleic acid and the analyte is a single stranded nucleic acid that has sufficient complementarity to macromolecule 120 to bind to the macromolecule. In some embodiments, the analyte is a single stranded nucleic acid that has sufficient complementarity to macromolecule 120 to bind to the macromolecule under conditions of high stringency (Section 6.27), intermediate stringency (Section 6.28), or low stringency (Section 6.29).

After exposure of macromolecule 120 to a solution that potentially includes an analyte, the biosensor is dried under, for example, nitrogen or argon. A voltage of between ±3 volts, between ±5 volts, or between ±7 volts is applied across electrically conducting materials 106 and 110 and the current is measured. Binding events are detected as difference in current across biological macromolecule before and after exposing the bound macromolecule 120 to a solution that potentially comprises an analyte. Representative analytes that may be used in this embodiment of the present invention are discussed in Section 6.14 below.

In one specific embodiment, materials 106 and 110 in a biosensor of the present invention are made of gold. Materials 106 and 110 are dried under nitrogen or argon. Materials 106 and 110 are connected to macro electrodes that are, in turn, connected to a voltage source. A voltage between ±3 volts, between ±5 volts, or between ±7 volts is applied. The materials 106 and 110 are then and tested for non-conductance, or a background conductance, using an EG&G high-speed potentiostat/galvanostat (e.g. Perkin-Elmer, Model 283). The biosensor is then contacted with a capture probe solution comprising derivatized oligonucleotides. The five prime end of the oligonucleotides is derivatized with a reactive group 122/masking group 124 comprising a thiocarbonate. For example, in some embodiments of the present invention, reactive group 122 is a thiol and masking group 124 is an S-2,2,2-trichloroethoxycarbonyl derivative that can be removed at −1.5 volts in the presence of LiClO₄/CH₃OH to reveal the thiol group 122. The reactive thiol group 122 can, in turn, form a covalent bond with an electrically conducting material (e.g., material 106 and/or 110), such as gold. The three prime end of the oligonucleotides is derivatized with a reactive group 125/masking group 126. In one example, the three prime end of the oligonucleotide includes a reactive thiol that is masked with a masking group such as an s-benzyloxycarbonyl derivative. The s-benzyloxycarbonyl derivative can be cleaved at −2.6 volts in N,N-dimethylformamide and tetrabutyl ammonium chloride to reveal the reactive thiol. In the method, material 106 is biased with the activation voltage 192 of the five prime electrolabile group (e.g., −1.5 volts) on the oligonucleotides thereby unmasking the thiol group (FIG. 1B). The unmasked thiol group attaches to material 106 (FIG. 1C). Then, material 110 is biased with the activation voltage 193 of the 3 prime electrolabile group of the capture probe (−2.6 volts) (FIG. 1D) thereby causing the nucleotide to bind to material 110 (FIG. 1E). Materials 106 and 110 are then dried again under nitrogen or argon. A voltage of between ±3 volts, between ±5 volts, or between ±7 volts is applied to the electrodes and the current is measured. The measured current of the hybridized nucleic acids is significantly greater then the current measured for the unhybridized electrodes.

It will be appreciated that, in various embodiments, the analyte can be hybridized to the macromolecule 120 at any stage during the method illustrated in FIG. 1. For example, the macromolecule 120 may be exposed to a solution that potentially comprises (e.g., is suspected of comprising) an analyte at the stage of the method illustrated by FIG. 1A (e.g., macromolecule 120 has not bound the first or second electrode), FIG. 1C (e.g., a first portion of macromolecule 120 has bound to the first electrode), or FIG. 1E (e.g., the macromolecule 120 spans the first and second electrode.).

6.2 Electrode Specific Biological Macromolecule Binding using an Intercalator

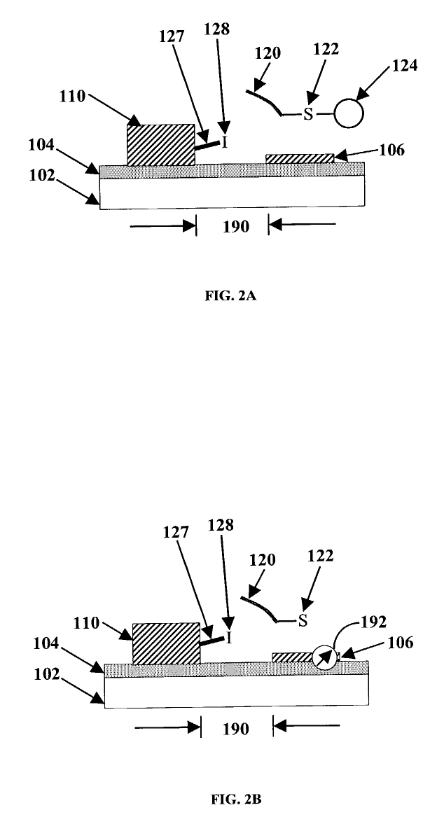

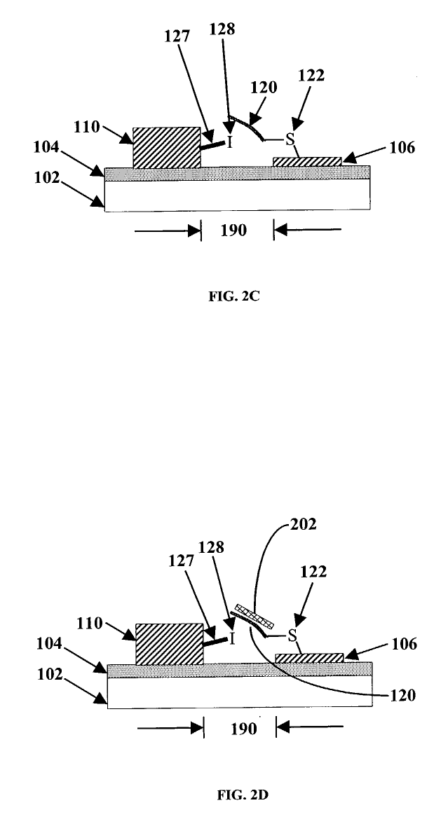

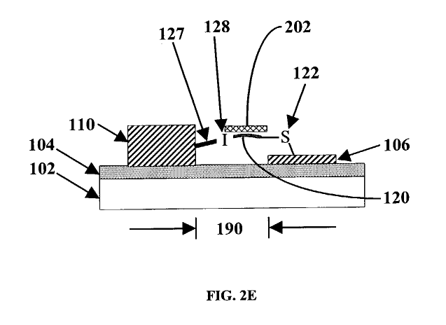

One aspect of the present invention make use of both an applied voltage and an intercalator to bind binding agents (e.g., macromolecules 120) to specific electrodes (e.g., electrically conducting materials 106 and 110) in an array of electrodes in a biosensor. FIGS. 2A through 2E illustrate one such a method in accordance with this aspect of the present invention.

FIG. 2A illustrates a biosensor that includes a substrate 102 and an optional insulator 104. Representative materials used to make substrate 102 are found in Section 6.9, below. Representative materials used to make optional insulator 104 are found in Section 6.11, below. The biosensor illustrated in FIG. 2A includes a first electrically conducting material 106 (e.g., a first electrode) and a second electrically conducting material 110 (e.g., a second electrode). Representative materials used to make electrically conducting materials 106 and 110 are found in Section 6.10, below. In embodiments where optional insulator 104 is not used, electrically conducting materials 106 and 110 overlay substrate 102.

In some embodiments in accordance with FIG. 2A, electrically conducting material 106 and electrically conducting material 110 are separated by a distance 190 that is 5 Angstroms or greater, 10 Angstroms or greater, 20 Angstroms or greater, 30 Angstroms or greater, or 100 Angstroms or greater. In some embodiments, a distance that is less than 100 Angstroms separates material 106 and material 110. In some embodiments, the distance between materials 106 and 110 is between 10 Angstroms to 100 Angstroms.

FIG. 2A also shows a macromolecule 120 (e.g., binding agent) that has been derivatized with reactive group S (FIG. 2A, element 122). Representative macromolecules 120 in accordance with the present invention are found in Section 6.13, below. Representative reactive groups 122 are found in Section 6.17, below. In FIG. 2A, reactive group 122 is masked by electrolabile masking group 124. Representative electrolabile masking groups 124 that can be used in this embodiment of the present invention are found in Section 6.17, below.

In FIG. 2A, a linker 127 and an intercalator 128 are bound to electrically conducting material 110. Methods disclosed in related U.S. application No. to be assigned, filed Dec. 26, 2002, titled “ASSOCIATION OF MOLECULES WITH ELECTRODES OF AN ARRAY OF ELECTRODES,” invented by Kunwar et al. and having attorney docket number 11210-019-999 and incorporated herein by reference in its entirety can be used to localize linker 127 with intercalator 128 to material 110. Electrically conducting materials 106 and 110 can be coated with a protective compound such as alkylsiloxane, an alkanetholate, and/or a fatty acid. Then, a voltage can be applied to material 110 in a device 144 in a plurality of devices 144 in a biosensor of the present invention, thereby stripping the protecting groups from the electrode (material 110). In the next step, linker 127 is exposed to the biosensor. In one embodiment, a first portion of linker 127 includes a reactive group (e.g., a sulfur group) that binds to the unprotected electrode (i.e., the unprotected material 110) and a second portion of linker 127 includes intercalator 128. Once linker 127 has bound to material 110, a voltage can be applied to material 106 to strip away the protective compound from material 106. The function of linker 127 and intercalator 128 will be described below in conjunction with FIGS. 2C and 2D.

In FIG. 2B, the method in accordance with this aspect of the invention continues with the application of a voltage 192 at electrically conducting material 106. Application of voltage 192 causes the electrolabile masking groups 124 to strip away from the macromolecules 120 that are in the vicinity of electrically conducting material 106. The stripping of electrolabile masking groups 124 from macromolecules 120 that are in the vicinity of electrically conducting material 106 exposes the reactive groups 122 on such macromolecules 120. The reactive groups 122 on such macromolecules 120, in turn, react with electrically conducting material 106 thereby binding such macromolecules 120 to electrically conducting material 106 as illustrated in FIG. 2C.

In one example, reactive group 122 and electrolabile masking group 124 together form a thiocarbonate. For example, in some embodiments of the present invention, reactive group 122 is a thiol and electrolabile masking group 124 is an S-2,2,2-trichloroethoxycarbonyl derivative that can be removed at −1.5 volts in the presence of LiClO₄/CH₃OH to reveal the thiol group 122. The reactive thiol group 122 can, in turn, form a covalent bond with electrically conducting material 106, thereby attaching macromolecule 120 to electrically conducting material 106. The binding of a macromolecule 120 to electrically conducting material 106 localizes the macromolecule 120 to a position near electrically conducting material 110 and, therefore, near intercalator 128.

After a macromolecule 120 has been bound to material 106, a solution that potentially comprises an analyte 202 is incubated with the biosensor for an amount of time. In some embodiments, this incubation period has a duration of less than one minute, less than five minutes, less than 15 minutes, less than 30 minutes, less than an hour, less than four hours, or less than one day. In some embodiments, the incubation period is between one second and one minute, between one minute and five minutes, between five minutes and fifteen minutes, between fifteen minutes and 30 minutes, between 30 minutes and one hour, or more than one hour. After the incubation period, the biosensor is washed to remove unbound analyte.

The proximity between intercalator 128 and bound macromolecule 120 allows intercalator 128 to intercalate the bound macromolecule 120 provided that analyte 202 has bound to macromolecule 120 during the incubation period. In some embodiments of the present invention, macromolecule 120 is a single stranded nucleic acid and analyte 202 is a nucleic acid that is complementary to macromolecule 120. In some embodiments, analyte 202 is a single stranded nucleic acid that binds to macromolecule 120 under conditions of high stringency (Section 6.27), conditions of intermediate stringency (Section 6.28), or conditions of low stringency (Section 6.29).

In some embodiments, intercalator 128 is highly selective and will only intercalate macromolecule 120 when the macromolecule 120 has bound to an analyte 202. In one example, macromolecule 120 is a single stranded nucleic acid that is localized to electrically conducting material 106 using the steps illustrated in FIGS. 2A through 2C. Then, the localized macromolecule 120 is exposed to a solution that potentially includes a single stranded nucleic acid that is complementary to macromolecule 120 (FIG. 2D). Because of this complementarity, the single stranded nucleic acid (analyte) and the localized macromolecule 120 form a double stranded nucleic acid. In this example, intercalator 128 only binds to double stranded nucleic acid. Thus, in this example, intercalator 128 serves as a sensor to molecular binding events. When localized macromolecule 120 binds to an analyte thereby forming double stranded nucleic acid, intercalator 128 intercalates macromolecule 120 thereby completing the electrical circuit between electrically conducting materials 106 and 110 (FIG. 2E). When localized biological macromolecule 128 does not bind to an analyte, double stranded nucleic acid is not formed, intercalator 128 does not intercalate macromolecule 120 and the electrical circuit between electrically conducting materials 106 and 110 is not formed. Representative intercalators 128 are disclosed in Section 6.18, below.

In some embodiments, intercalator 128 only intercalates macromolecule 120 when biological macromolecule binds to a specific type of analyte 202. For example, in some embodiments, macromolecule 120 is single stranded nucleic acid and intercalator 128 only intercalates macromolecule 120 when macromolecule 120 binds to an RNA that is complementary to all or a portion of macromolecule 120. In another example, macromolecule 120 is single stranded nucleic acid and intercalator 128 only intercalates macromolecule 120 when macromolecule 120 binds to a single stranded DNA that is complementary to all or a portion of macromolecule 120. In some embodiments, intercalator 128 is ethidium bromide, acridine, or derivatives thereof.

Although specific examples of electrolabile masking groups 124, reactive groups 122, and voltages 192 are described in this section, the present invention is not limited to such examples. Indeed, any set of masking groups 124 that are electrically labile at voltage may be used in the present invention. Representative electrolabile masking groups 124 and reactive groups 122 that may be used in this embodiment of the present invention are disclosed in Section 6.17, below.

In one example, the method of the present invention described above and illustrated in FIGS. 2A-2E is used to attach a macromolecule 120 to a biosensor. Next, electrically conducting materials 106 and 110 are connected to a voltage source and tested for non-conductance, or a background conductance, prior to exposing the bound macromolecule 120 to an analyte 202. Background conductance is measured, using, for example, a high-speed potentiostat/galvanostat (e.g. Perkin-Elmer, Model 283). Then, the bound macromolecule 120 is exposed to a solution that may comprise an analyte 202. In one example, macromolecule 120 is a single stranded nucleic acid and the analyte 202 is a single stranded nucleic acid that has sufficient complementarity to macromolecule 120 to bind to the macromolecule.

After exposure of macromolecule 120 to a solution that potentially includes an analyte, the biosensor is dried under, for example, nitrogen or argon. A voltage of between ±3 volts, between ±5 volts, or between ±7 volts is applied across electrically conducting materials 106 and 110 and the current is measured. Binding events are detected as difference in current across macromolecule 120 before and after exposing the bound macromolecule 120 to a solution that potentially comprises an analyte 202. Representative analytes 202 that may be used in this embodiment of the present invention are discussed in Section 6.14, below.

The embodiment illustrated in FIG. 2A represents the case in which a reactive group 122 is used to localize a macromolecule 120 to an electrode pair (e.g., electrically conducting material 106 and 110) in a biosensor. Then, an intercalator 128 is used to complete an electrical circuit in the electrode pair.

One embodiment in accordance with the present invention provides a method of electrically coupling an electrode pair in a plurality of electrode pairs in a biosensor with a macromolecule 120. The electrode pair comprises a first electrode and a second electrode (e.g., electrically conducting material 106 and electrically conducting material 110). An intercalator is covalently linked to the second electrode. A portion of the macromolecule 120 is derivatized with a reactive group that is masked with an electrolabile masking group. In this embodiment, a voltage is applied at the first electrode in the electrode pair under conditions that are sufficient to unmask the reactive group. The unmasked reactive group binds to the first electrode thereby linking the macromolecule 120 to the first electrode. The electrode pair is then exposed to a solution that potentially comprises an analyte for a period of time. When the analyte binds to the macromolecule to form a complex comprising the macromolecule and the analyte, the intercalator binds to the complex thereby electrically connecting the electrode pair. In this exemplary embodiment, all or a portion of macromolecule 120 is a single stranded nucleic acid and the analyte that binds to macromolecule 120 is a single stranded nucleic acid that binds to a portion of macromolecule 120 thereby forming a double stranded nucleic acid complex comprising the macromolecule and the analyte. Furthermore, in this example, the intercalator intercalates the double stranded nucleic acid complex comprising the macromolecule and the analyte thereby electrically connecting the electrode pair.

6.3 Electrode Specific Biological Macromolecule Binding using an Electrolabile Masking Group and a Photosensitive or Chemically Sensitive Masking Group

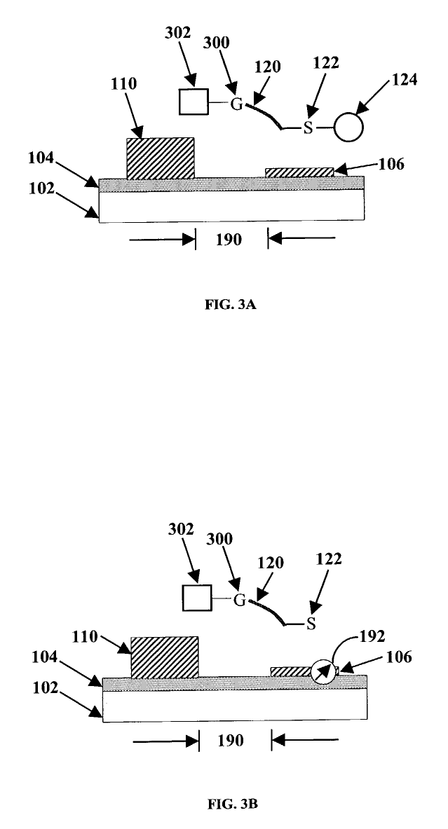

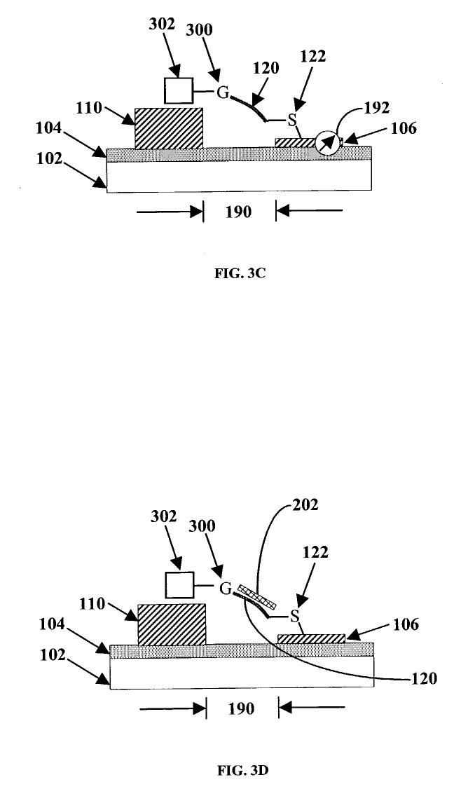

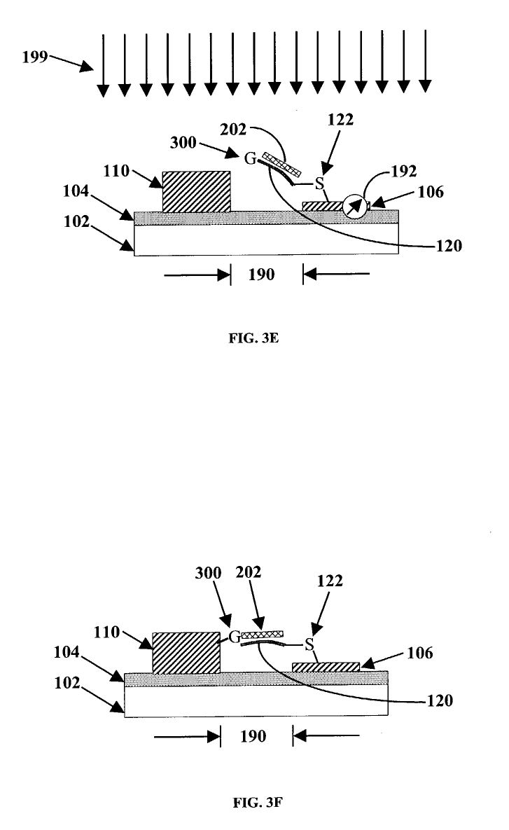

One aspect of the present invention make use of both an applied voltage and a photosensitive or chemcially sensitive group to bind binding agents (e.g., biological macromolecules 120) to specific electrodes (e.g., electrically conducting materials 106 and 110) in an array of electrodes in a biosensor. FIGS. 3A through 3F illustrate one such a method in accordance with this aspect of the present invention.

FIG. 3A illustrates a biosensor that includes a substrate 102 and an optional insulator 104. Representative materials used to make substrate 102 are found in Section 6.9, below. Representative materials used to make optional insulator 104 are found in Section 6.11, below. The biosensor illustrated in FIG. 3A includes a first electrically conducting material 106 (e.g., a first electrode) and a second electrically conducting material 110 (e.g., a second electrode). Representative materials used to make electrically conducting materials 106 and 110 are found in Section 6.10, below. In embodiments where optional insulator 104 is not used, electrically conducting materials 106 and 110 overlay substrate 102.

In some embodiments in accordance with FIG. 3A, electrically conducting material 106 and electrically conducting material 110 are separated by a distance 190 that is 5 Angstroms or greater, 10 Angstroms or greater, 20 Angstroms or greater, 30 Angstroms or greater, or 100 Angstroms or greater. In some embodiments, a distance that is less than 100 Angstroms separates material 106 and material 110. In some embodiments, the distance between materials 106 and 110 is between 10 Angstroms to 100 Angstroms.

FIG. 3A also shows a macromolecule 120 (e.g., binding agent). A first portion of macromolecule 120 has been derivatized with reactive group S (FIG. 3A, element 122). Representative macromolecules 120 are found in Section 6.13, below. Representative reactive groups 122 are found in Section 6.17, below. In FIG. 3A, reactive group 122 is masked by electrolabile masking group 124. Representative electrolabile masking groups 124 that can be used in this embodiment of the present invention are found in Section 6.17, below.

As illustrated in FIG. 3A, second portion of biological macromolecule 120 is derivatized with a chemically reactive group 300 that is masked by a photosensitive or chemically sensitive masking group 302. The function of chemically reactive group 300 and photosensitive or chemcially sensitive masking group 302 will be described below in conjunction with FIGS. 3C and 3D.

In one example in accordance with the present invention, reactive group 122 is a thiol and electrolabile masking group 124 is an S-2,2,2-trichloroethoxycarbonyl derivative that can be removed at −1.5 volts in the presence of LiClO₄/CH₃OH to reveal the thiol group 122. The reactive thiol group 122 can, in turn, form a covalent bond with an electrically conducting material (e.g., material 106 and/or 110), such as gold. In another example in accordance with the present invention, reactive group 122 is a thiol and electrolabile masking group 124 is an s-benzyloxycarbonyl derivative. The s-benzyloxycarbonyl derivative can be removed from the reactive thiol at −2.6 volts in the presence of DMF and tetrabutyl ammonium chloride. This reaction exposes a reactive sulfur group 122 that can bind with an electrically conducting material such as gold.

In FIG. 3B, the method in accordance with this aspect of the invention continues with the application of a voltage 192 at electrically conducting material 106. Application of voltage 192 causes the electrolabile masking groups 124 to strip away from the macromolecules 120 that are in the vicinity of electrically conducting material 106. The stripping of electrolabile masking groups 124 from macromolecules 120 that are in the vicinity of electrically conducting material 106 exposes the reactive groups 122 on such macromolecules 120. The reactive groups 122 on such macromolecules 120, in turn, react with electrically conducting material 106 thereby binding such macromolecules 120 to electrically conducting material 106 as illustrated in FIG. 3C.

In one example, reactive group 122 and electrolabile masking group 124 together form a thiocarbonate. For example, in some embodiments of the present invention, reactive group 122 is a thiol and electrolabile masking group 124 is an S-2,2,2-trichloroethoxycarbonyl derivative that can be removed at −1.5 volts in the presence of LiClO₄/CH₃OH to reveal the thiol group 122. The reactive thiol group 122 can, in turn, form a covalent bond with electrically conducting material 106, thereby attaching macromolecule 120 to electrically conducting material 106.

After a macromolecule 120 has been bound to material 106, a solution that potentially comprises an analyte 202 is incubated with the biosensor for an amount of time. In some embodiments, this incubation period has a duration of less than one minute, less than five minutes, less than 15 minutes, less than 30 minutes, less than an hour, less than four hours, or less than one day. In some embodiments, the incubation period is between one second and one minute, between one minute and five minutes, between five minutes and fifteen minutes, between fifteen minutes and 30 minutes, between 30 minutes and one hour, or more than one hour. After the incubation period, the biosensor is washed to remove unbound analyte.

After unbound analyte 202 has been removed, the biosensor is exposed to a light source or a chemical source 199 (FIG. 3E) in order to remove mask 302, thereby exposing reactive group 300. In some embodiments, source 199 is applied in a global fashion. That is, different derivatized macromolecules 120 are attached to different electrode pairs in the biosensor using the methods described above. Each bound macromolecule 120 is incubated with analyte 202. Then source 199 (chemical or electrical) is globally applied to the biosensor so that each masking group 300 is stripped from the respective macromolecule 120 at the same time. In some embodiments, only a portion of the biosensor, and therefore only a portion of the electrode pairs of the biosensor, are exposed to light.

As illustrated in FIG. 3F, removal of masking group 302 allows reactive group 300 to bind to electrically conducting material 110, thereby completing the electrical circuit between electrically conducting materials 106 and 110.

In some embodiments of the present invention, macromolecule 120 is a single stranded nucleic acid and analyte 202 is a nucleic acid that is complementary to macromolecule 120. In some embodiments, analyte 202 is a single stranded nucleic acid that binds to macromolecule 120 under conditions of high stringency (Section 6.27), conditions of intermediate stringency (Section 6.28), or conditions of low stringency (Section 6.29).

In one embodiment of the present invention, electrically conducting materials 106 and 110 are connected to a voltage source and tested for non-conductance, or a background conductance, prior to localizing a macromolecule 120 to an electrode pair (e.g., materials 106 and 110) using the methods illustrated in FIG. 3. Background conductance is measured, using, for example, a high-speed potentiostat/galvanostat (e.g. Perkin-Elmer, Model 283). Then, after the method illustrated in FIGS. 3A through 3F have been performed, the biosensor is dried under, for example, nitrogen or argon gas. A voltage of between ±3 volts, between ±5 volts, or between ±7 volts is applied across electrically conducting materials 106 and 110 and the current is measured. Binding events are detected as difference in current across macromolecule 120 before and after localizing macromolecule 120 to an electrode pair using the method illustrated in FIG. 3.

Representative analytes 202 that may be used in this embodiment of the present invention are discussed in Section 6.14, below. Although specific examples of electrolabile masking groups 124, reactive groups 122, and voltages 192 are described in this section, the present invention is not limited to such examples. Indeed, any set of masking groups 124 that are electrically labile at voltage may be used in the present invention. Representative electrolabile masking groups 124 and reactive groups 122 that may be used in this embodiment of the present invention are disclosed in Section 6.17, below. Representative photosensitive masking groups 302 and chemically sensitive masking groups 302 as well as reactive groups 300 are disclosed in Section 6.19, below.

It will be appreciated that, in alternate embodiments some of which are not illustrated, the analyte can be hybridized to the macromolecule 120 at any stage during the method illustrated in FIG. 3. For example, the macromolecule 120 may be exposed to a solution that potentially comprises (e.g., is suspected of comprising) an analyte at the stage of the method illustrated by FIG. 3A (e.g., macromolecule 120 has not bound the first or second electrode), FIG. 3C (e.g., a first portion of macromolecule 120 has bound to the first electrode), or FIG. 3F (e.g., the macromolecule 120 spans the first and second electrode.).

In another embodiment in accordance with the present invention, electrode pairs of a biosensor 100 are cleaned to remove organic contaminants. A layer of protective molecules is associated with each electrode, preferably by contacting electrode pairs of the array with a liquid comprising one or more protective molecules. The protective layer of molecules preferably comprises at least one of an alkylsiloxane, an alkanethiolate, and a fatty acid. Next, a first electrode of each electrode pair is associated with a first macromolecule 120. The association is preferably performed by contacting electrode pairs of the array with at least one liquid comprising at least one first macromolecule 120 to be associated with an electrode of at least one electrode pair. A first macromolecule 120 may be associated with an electrode by deprotecting the electrode. Electrode deprotection is preferably performed by modifying an electrical potential of an electrode with respect to a reference electrode. See, for example, U.S. application No. to be assigned, filed Dec. 26, 2002, titled “ASSOCIATION OF MOLECULES WITH ELECTRODES OF AN ARRAY OF ELECTRODES,” invented by Kunwar et al. and having attorney docket number 11210-019-999 and incorporated herein by reference in its entirety. In one example, a device in a plurality of devices is contacted with a liquid comprising a macromolecule 120. Upon deprotecting the first electrode 106 in the device 144, macromolecule 120 associates with electrode 106, preferably via a first portion reactive group on the macromolecule 120. Protective molecules associated with the second electrode (electrically conducting material 110) in the device 144 inhibit association of macromolecule 120 with the second electrode. In this embodiment, macromolecule 120 includes an unmasked first reactive group (e.g., 122 without mask 124, FIG. 3A) as well as a second reactive group (e.g., 300 of FIG. 3A) that is masked by voltage sensitive leaving group, a chemically sensitive leaving group, or a light sensitive leaving group.

Subsets of electrode pairs of a biosensor may be contacted with respective liquids, each comprising at least one different macromolecule 120 to be associated with an electrode of an electrode pair 144 of the subset of electrode pairs. For example, a device 144-N^{a} may be contacted with a liquid comprising a macromolecule 120. Upon deprotecting electrode 106-N^{a}, macromolecule 120 associates with the electrode, preferably through the first reactive group on a first portion of the macromolecule 120. Protective molecules associated with electrode 110-N^{a} inhibit association of macromolecule 120 with electrode 110-N^{a} at this stage. By contacting subsets of electrode pairs with respective liquids, each comprising a respective different first molecule, a plurality of different electrodes may each be associated with a different macromolecule 120 in less time than would be required to contact all electrode pairs with a liquid comprising the same molecule and deprotecting only one electrode of the array during each contacting step.

In subsequent steps, the protective layer is removed from the second electrode 110 in each device 144. However, macromolecule 120 does not bind to the unmasked second electrode (electrode 110) because the second reactive group on macromolecule 120 is still masked by a protecting group. At this stage, the protecting group is removed from the macromolecule 120 by exposing the macromolecule 120 to the appropriate chemical, light source or voltage. Upon removal of the protecting group, the second reactive group binds to the second electrode thereby causing the macromolecule 120 to span the first and second electrode.

The macromolecule 120 can be incubated with analyte at any stage of the methods of the present invention. In some embodiments, macromolecule 120 is incubated with an analyte after the macromolecule 120 has been bound to the first electrode but not the second electrode. In some embodiments, macromolecule 120 is incubated with analyte after the macromolecule 120 spans the first and second electrode in a device in the plurality of devices. In some embodiments, (i) the macromolecule 120 is bound to the first and second electrodes, (ii) a reference electrical measurement is taken across the first and second electrodes, (iii) the bound macromolecule 120 is exposed to the analyte under hybridizing conditions, and (iv) a any difference in the electrical measurement across the first and second electrodes is determined.

6.4 Binding Multiple Macromolecules to a Plurality of Electrode Pairs

In some embodiments of the present invention, the methods that are disclosed in Sections 6.1, 6.2, and 6.3 are used to populate a plurality of electrode pairs in a biosensor with macromolecules 120. An electrode pair is considered populated when a macromolecule 120 is used to form a connection between a first and second electrode in the electrode pair as illustrated in FIGS. 1E, 2E, and 3F. In some embodiments, each electrode pair in the biosensor is populated with a different macromolecule 120. In some embodiments, each electrode pair in the biosensor is populated with the same macromolecule 120. In still other embodiments, the same type of macromolecule 120 is used to populate a portion of the electrode pairs in a biosensor.

The methods that are disclosed in Sections 6.1, 6.2, and 6.3 may be repeated in order to populate each electrode pair in a biosensor with a different macromolecule 120. For example, consider the case where electrolabile masking groups 124 and 126 are used to direct a macromolecule 120 to an electrode pair in accordance with Section 6.1. In the method, voltages 192 and 193 are applied at a specific electrode pair (e.g., materials 106 and 110) in order to populate the electrode pair with a macromolecule 120. After the specific electrode pair has been populated, the biosensor can be washed and exposed to a different macromolecule 120. Then, the steps in Section 6.1 can be repeated using a different electrode pair (e.g., a different set of materials 106 and 110). Similarly, the methods disclosed in Section 6.2 can be used to populate a plurality of electrode pairs by using a washing step each time a particular electrode pair or group of electrode pairs has been populated with a macromolecule 120.

The methods disclosed in Section 6.3 can also be used to populate a plurality of electrode pairs with different biological macromolecules 120 by washing the biosensor each time the methods disclosed in Section 6.3 are repeated. Furthermore, the light (or chemical) exposure step disclosed in FIG. 3E can be postponed until each of the desired biological macromolecules 120 has been localized to the desired respective first electrodes (e.g., to respective materials 106) in the biosensor. After each of the desired biological macromolecules 120 has been localized to the desired respective first electrodes, a source 199 (e.g., light or chemical) can be applied thereby removing the masking groups 302 on each respective biological macromolecule 133 in the biosensor in the same step.

6.5 Illustrative Biosensors

The methods disclosed in Sections 6.1 through 6.4, above, are not limited to the biosensor configurations disclosed in FIGS. 1, 2, and 3. This section describes a number of biosensor configurations that can be used in accordance with the methods disclosed in Sections 6.1 through 6.4. In addition to the biosensor configurations disclosed in this section, additional biosensor configurations that may be used in accordance with the present invention are described in U.S. patent application Ser. No. 09/970,087, filed Oct. 2, 2001, U.S. Provisional Patent Application No. 60/297,583, filed on Jun. 11, 2001, and U.S. Provisional Patent Application No. 60,372,933, filed on Apr. 17, 2002 each of which is incorporated herein, by reference, in their entireties. In addition, any biosensor configuration described in copending U.S. application No. to be assigned, filed Dec. 26, 2002, titled “DEVICE STRUCTUR FOR CLOSELY SPACED ELECTRODES,” invented by Kunwar et al. and having attorney docket number 11210-018-999 and incorporated herein by reference in its entirety, can be used in the methods of the present invention. Although materials 106 and 110 appear to have different thickness in FIGS. 1, 2, and 3, in some embodiments of the present invention, materials 106 and 110 in biosensors in accordance with FIGS. 1, 2, and 3 have the same thickness. In some embodiments of the present invention, the biosensor comprises a plurality of devices and each device in the plurality of devices includes an electrode pair that is separated by a molecular distance such that the electrode pair can be spanned by a macromolecule. That is, a first portion of a macromolecule can bind to a first electrode in an electrode pair in the biosensor and a second portion of a macromolecule can bind to a second electrode in an electrode pair in the biosensor.

6.5.1. Illustrative Biosensor with Non-Overlapping Electrodes

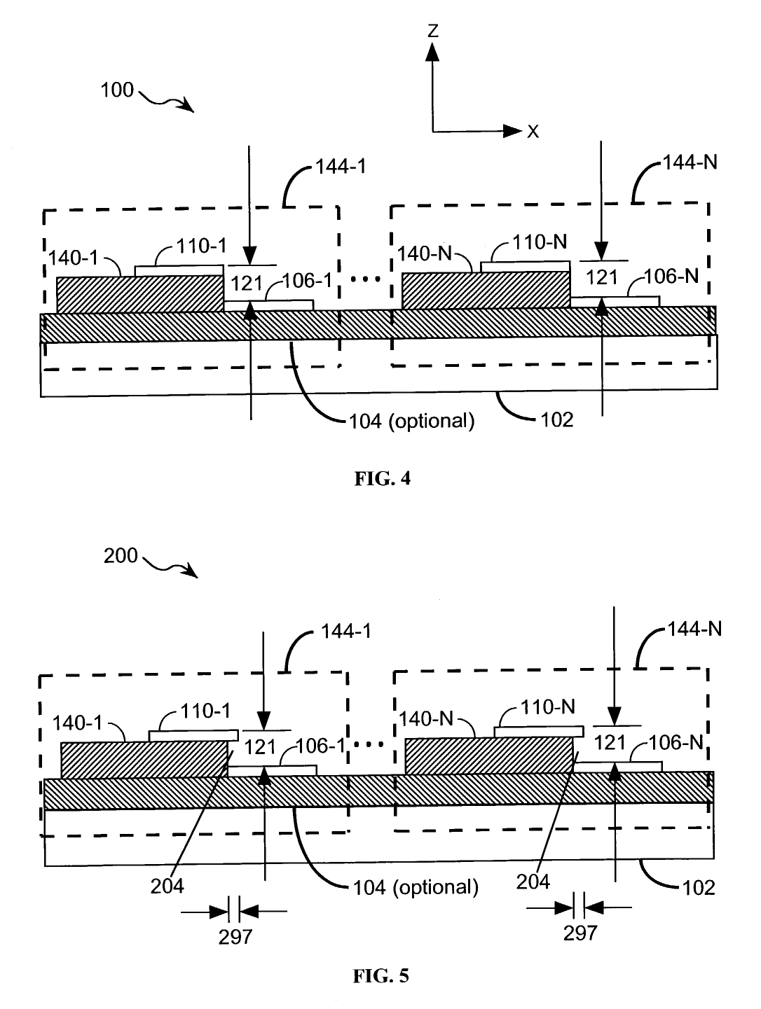

FIG. 4 illustrates a side plan view of a novel biosensor 100 that can be used in the methods disclosed in Sections 6.1 through 6.4. Biosensor 100 includes non-overlapping electrically conducting materials 106 and 110. In some embodiments, a predetermined distance 121 in the z-dimension separates the top of electrically conducting material 106 and the top of electrically conducting material 110. In some embodiments, electrically conducting materials 106 and 110 are made of conductive, semi-conductive, or resistive materials. In some embodiments, predetermined distance 121 is achieved by overlaying electrically conducting material 110 on a spacer 140.

As illustrated in FIG. 4, spacer 140 and electrically conducting materials 106 and 110 comprise a discrete device 144. In instances where electrically conducting materials 106 and 110 are electrodes, each device 144 has an electrode-insulator-electrode configuration. It will be appreciated that each device 144 may serve as an independent sensor for a particular application. It will be appreciated that each device has an electrically conducting material 106 and an electrically conducting material 110 that, together, is referred herein as an electrode pair.

An advantage of the present invention is that predetermined distance 121 can be precisely controlled by separating electrically conducting materials 106 and 110 in the z dimension (FIG. 4) rather than the x dimension or the y dimension (not shown, perpendicular to the plane of FIG. 4). Separation in the z dimension is controlled using precise semiconductor manufacturing techniques that are described in copending U.S. application No. to be assigned, filed Dec. 26, 2002, titled “DEVICE STRUCTUR FOR CLOSELY SPACED ELECTRODES,” invented by Kunwar et al. and having attorney docket number 11210-018-999 and incorporated herein by reference in its entirety.

In one embodiment of the present invention, electrically conducting materials 106 and 110 are electrodes. A macromolecule 120, or pool of macromolecules 120, can be directly bound to electrodes 106 and 110 as described in Sections 6.1 through 6.4, above. Generally speaking, macromolecules 120 are attached to electrodes 106 and 110 in such a manner that sufficient area on the macromolecule 120 is left so that the macromolecule 120 can bind with its “cognate” target molecule. When a macromolecule 120 binds to its cognate target molecule, a binding agent/target molecule complex is formed whose conductivity is different than the conductive of macromolecule 120 alone. This change in conductivity is readily detected indicating the presence and/or concentration of macromolecule 120 on the biosensor (e.g., on biosensor 100).

Referring to FIG. 4, one embodiment of the present invention provides a biosensor 100 comprising a plurality of devices 144 on a substrate 102. Each device 144 in the plurality of devices 144 occupies a different region on an optional insulator layer 104. Optional insulator layer 144 is overlaid on substrate 102. Furthermore, each device 144 in the plurality of devices comprises (i) a first electrically conducting material 106 having a top surface, wherein the first electrically conducting material 106 is overlaid on a first portion of optional insulator layer 104, (ii) a spacer 140 overlaid on a second portion of the insulator layer 104, and (iii) a second electrically conducting material 110 overlaid on a portion of spacer 144. As illustrated in FIG. 4, the first electrically conducting material 106 and spacer 144 abut each other. Furthermore, for any given device 144 in the plurality of devices, the first portion of insulator layer 104 occupied by the device does not overlap with the second portion of insulator layer 104 occupied by the device. As used herein, a device 144 “occupies” that portion of insulator layer 104 which is overlaid by a component (e.g., material 106, spacer 140, etc.) of the device. In embodiments where insulator 104 is not used, each device 144 occupies a portion of substrate 102 and material 106 and spacer 140 each directly overlay a portion of substrate 102.

In some embodiments in accordance with FIG. 4, a distance between a plane including the top surface of the first electrically conducting material 106 and a plane including the top surface of the second electrically conducting material 110 is less than 500 Angstroms. In some embodiments of the present invention, the distance between a plane including the top surface of the first electrically conducting material 106 and a plane including the top surface of the second electrically conducting material 110 is less than 250 Angstroms. In still other embodiments, a distance between a plane including the top surface of the first electrically conducting material and a plane including the top surface of the second electrically conducting material is less than 100 Angstroms. In still other embodiments of the present invention, a distance between a plane including the top surface of the first electrically conducting material 106 and a plane including the top surface of the second electrically conducting material 110 is between about 40 Angstroms and about 60 Angstroms, or between about 20 Angstroms and 100 Angstroms.

6.5.2 Illustrative Biosensor with Overlapping Electrodes

FIG. 5 illustrates a side plan view of a novel biosensor 200 in accordance with another embodiment of the present invention. Biosensor 200 is similar to biosensor 100 (FIG. 4) with the exception that materials 106 and 110 overlap each other. As illustrated in FIG. 5, materials 106 and 110 overlap, thereby creating a cavity 204. Furthermore, in the embodiment illustrated in FIG. 5, there is no composition in cavity 204, such as spacer 140 or insulator layer 104.

The width 297 of cavity 204 defines the amount that materials 106 and 110 overlap in biosensor 200 (FIG. 5). In some embodiments of the present invention, cavity 204 has a width 297 that is 300 Angstroms or less, 250 Angstroms or less, 200 Angstroms or less, 150 Angstroms or less, 100 Angstroms or less, 50 Angstroms or less, or Angstroms or less. In some embodiments of the present invention, cavity 204 has a width 297 that is between 10 Angstroms and 100 Angstroms.

6.5.3 Illustrative Biosensor with Cavity in the Insulator Layer

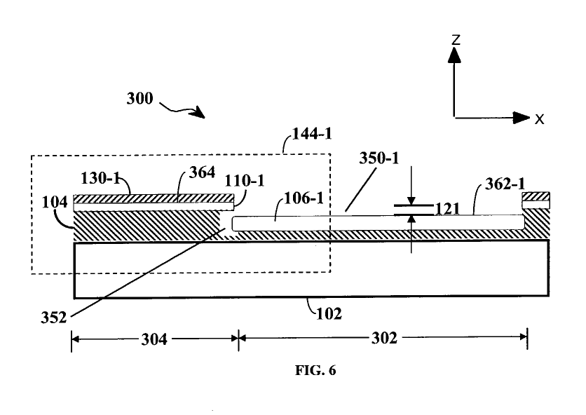

FIG. 6 illustrates a side plan view of a biosensor 300 in accordance with another embodiment of the present invention. Biosensor 300 includes substrate 102. Insulator layer 104 overlays substrate 102. As illustrated in FIG. 6, cavity 350 is introduced into portion 302 of insulator layer 104 and material 106 is deposited in cavity 350. Further, material 110 is overlaid on portion 304 of insulator layer (FIG. 6). In this way, insulator layer 104 is used to separate material 106 from material 110 in the z dimension. Finally, optional passivator 130 is overlaid on material 110 to complete device 144. Biosensor 300 (FIG. 6) differs from biosensors 100 and 200 in the sense that biosensor 300 does not use a spacer 140 to separate material 106 from material 110. Rather, in biosensor 300, desired separation between material 106 and 110 is achieved by the formation of cavity 350.

Some embodiments of the present invention provide a biosensor comprising a plurality of devices 144 on a substrate 102 (FIG. 6). Each device in the plurality of devices occupies a different region on an insulator layer 104. The insulator layer 104 is overlaid on substrate 102 and each device 144 in the plurality of devices 144 is associated with a different cavity 350 in the insulator layer. Only one such device 144 is shown in FIG. 6. However, biosensor 300 may have any number of devices 144 and each such device 144 includes a cavity 350. Each device 144 in the plurality of devices 144 of biosensor 300 comprise (i) a first electrically conducting material 106 having a top surface 362, wherein material 106 is overlaid in the different cavity 350 associated with the device 144, (ii) a second electrically conducting material 110 having a top surface 364, wherein material 110 is overlaid on insulator 104 in a region outside of the cavity 350 associated with the device 144; and (iii) a passivation layer 130 overlaid on material 110.

Referring again to FIG. 6, in some embodiments of the present invention, an additional cavity 352 is etched into insulator layer 104 to further isolate material 106 from material 110. In some embodiments of the present invention, each cavity 350 in biosensor 300 has a width 302 between 100 Angstroms and 300 Angstroms, a width 302 between 200 Angstroms and 800 Angstroms, a width 302 between 500 Angstroms and 2000 Angstroms, or a width 302 that is greater than 5000 Angstroms. In some embodiments of the present invention, each cavity 350 in biosensor 300 has a width that is about two microns or larger, about ten microns or larger, or about twenty-five microns or larger.

6.5.4 Additional Biosensor Configurations

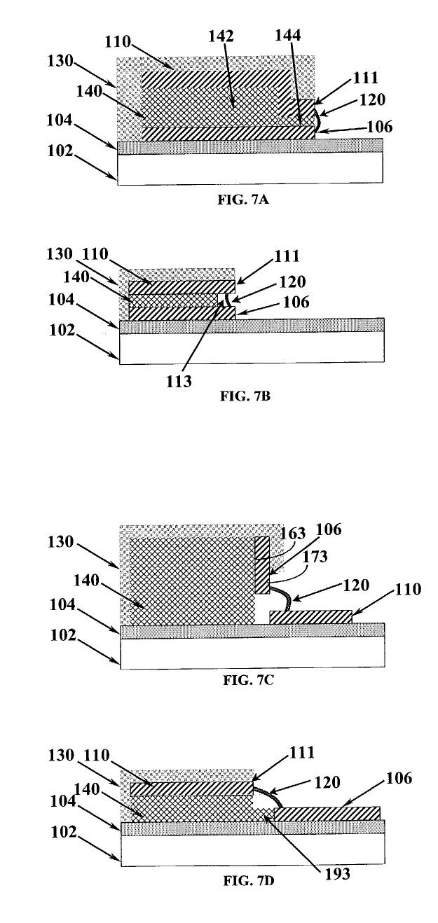

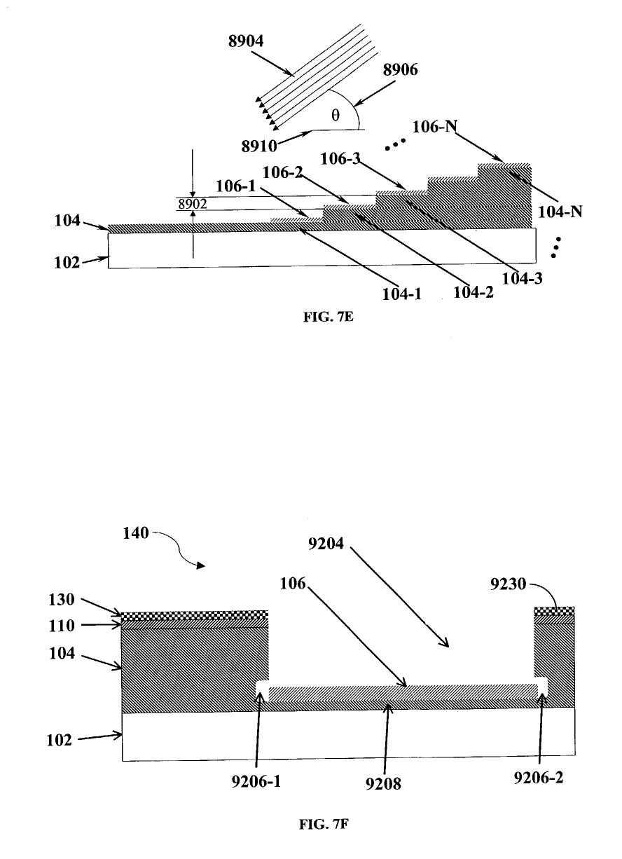

Some embodiments of biosensors (e.g. biosensors 100, 200 and 300) in accordance with the present invention have been described. Attention now turns to FIGS. 7A through 7F, which illustrate plan views of several biosensors in accordance with additional embodiments of the present invention. Although only a single device 144 is shown in the biosensor configurations illustrated in FIGS. 7A through 7F, it will be appreciated that any number of devices 144 may be found in the biosensors illustrated in FIGS. 7A through 7F.

Attaching specific entities (e.g. macromolecules) at locations on materials 106 and 110 in biosensors 100, 200, 300, or the biosensors illustrated in FIGS. 7A through 7F may be used either to bridge the entity between materials 106 and 110 or to localize the entities for further reactions. In the case where entities are bridged, one end of the entity may be attached to, for example, material 106-1 while another end of the entity may be attached to, for example, material 110-1 (FIG. 4). In the case where materials 106 and 110 are electrodes, such a bridging configuration can be used as a biosensor. That is, changes in the electrical conductivity of the entity can be precisely measured. Such measurements may be used to detect when a foreign object binds to the bridged entity. As such, the biosensors of the present invention may be used as a sensor of molecular events. Such sensors can be used in many fields including, but not limited to, biology, chemistry, physics, genomics and proteomics.

The biosensor illustrated in FIG. 7A includes a substrate 102 and an optional insulator 104 overlaid on substrate 102. In the case where optional insulator 104 is not used, material 106 is directly overlaid on substrate 102 (not shown). In the case where optional insulator 104 is used, material 106 is overlaid onto a portion of insulator 104. Next, a spacer 140 is overlaid on material 106. Spacer 140 has two segments, a thick segment 142 and a thin segment 144. The thickness of thin segment 142 defines a separation distance between material 106 and material 110, which is overlaid on spacer 140. Further, passivation layer 130 overlays all exposed surfaces of material 110 except sidewall 111. In some embodiments, passivation layer 130 helps cause macromolecule 120 to span from sidewall 111 of material 110 to the exposed sidewall of material 106.

Referring again to FIG. 7A, one embodiment of the present invention provides a biosensor that includes a plurality of devices 144 on a substrate 102. Each device 144 in the plurality of devices 144 occupies a different region on an insulator layer 104. Furthermore, each device 144 in the plurality of devices 144 is capable of binding to a macromolecule 120. In this biosensor, the insulator layer 104 is overlaid on the substrate 102. At least one device 144 in the plurality of devices comprises (i) a first electrically conducting material 106, (ii) a spacer 140 overlaying the first electrically conducting material 106, (iii) a second electrically conducting material 110 overlaid on the spacer 140, and (iv) a passivation layer 130 overlaid on the second electrically conducting material 110. In this device, the first electrically conducting material 106 is overlaid on the different region of the insulator layer occupied by the device 144. Further, the spacer 140 includes a step region 144 and a main region 142 and the step region 144 of the spacer 140 is not as thick as the main region 142 of the spacer 140.

The biosensor illustrated in FIG. 7B includes a substrate 102 and an optional insulator 104 overlaid on substrate 102. In the case where optional insulator 104 is not used, material 106 is overlaid on substrate 102 (not shown). In the case where optional insulator 104 is used, material 106 is overlaid onto a portion of insulator 104. Next, a spacer 140 is overlaid onto a portion of material 106. The thickness of spacer 140 defines a separation distance between material 106 and material 110, which is overlaid on spacer 140. Further, a passivation layer 130 overlays all exposed surfaces of material 110 except sidewall 111 of material 110. In some embodiments, passivation layer 130 causes macromolecule 120 to span from sidewall 111 of material 110 to the exposed sidewall of material 106.

Referring again to FIG. 7B, one embodiment of the present invention provides a biosensor comprising a plurality of devices 144 on a substrate 102. Each device 144 in the plurality of devices 144 occupies a different region on the substrate 102 and each device 144 in the plurality of devices 144 is capable of binding to a macromolecule 120. At least one device in the plurality of devices comprises (i) a first electrically conducting material, wherein the first electrically conducting material is overlaid on the different region of the substrate 102 occupied by the device 144, (ii) a spacer 140 overlaying the first electrically conducting material 106, (iii) a second electrically conducting material on the spacer 140 so that a cavity 113 is formed, and (iv) a passivation layer 130 overlaid on the second electrically conducting material.

The biosensor illustrated in FIG. 7C includes a substrate 102 and an optional insulator 104 overlaid on substrate 102. In the case where optional insulator 104 is not used, material 110 is overlaid on a first portion of substrate 102 and spacer 140 is overlaid on a second portion of substrate 102. In the case where optional insulator 104 is used, material 110 is overlaid onto a first portion of insulator 104 and spacer 140 is overlaid on a second portion of insulator 104. Spacer 140 includes a sidewall 163 and material 106 is overlaid on a portion of sidewall 163. Passivation layer 130 is overlaid on spacer 140 and a portion of material 106. In some embodiments, macromolecule 120 spans between surface 173 of material 106 and the upper surface of material 110.

Referring again to the biosensor illustrated in FIG. 7C, one embodiment of the present invention provides a biosensor comprising a plurality of devices 144 on a substrate 102. Each device 144 in the plurality of devices 144 occupies a different region on an insulator layer 104. Furthermore, each device 144 in the plurality of devices 144 is capable of binding to a macromolecule 120. Insulator layer 104 is overlaid on substrate 102. Each device 144 in the plurality of devices 144 comprises (i) an electrically conducting material 110, (ii) a spacer overlaid on a second portion of the different region of insulator layer 104 occupied by device 144, (iii) an electrically conducting material 106 that abuts side-wall 163 of spacer 140; and (iv) a passivation layer 130 that overlays spacer 140 and a portion of electrically conducting material 106. In this embodiment, electrically conducting material 110 is overlaid on a first portion of the different region of the insulator layer 104 occupied by the device 144. Furthermore, the first portion of insulator layer 104 does not overlap with the second portion of insulator layer 104.

The biosensor illustrated in FIG. 7D includes a substrate 102 and an optional insulator 104 overlaid on substrate 102. In the case where optional insulator 104 is not used, material 106 is overlaid on a first portion of substrate 102 and spacer 140 is overlaid on a second portion of substrate 102, where the second portion of substrate 102 is adjacent to the first portion of substrate 102 (not shown). In the case where optional insulator 104 is used, material 106 is overlaid onto a first portion of insulator 104 and spacer 140 is overlaid on a second portion of insulator 104, where the second portion of insulator 104 is adjacent to the first portion of insulator 104. Next, material 110 is overlaid on spacer 140 and passivation layer 130 is overlaid on all exposed portions of material 110 except sidewall 111. In some embodiments, macromolecule 120 spans between sidewall 111 of material 110 and the upper surface of material 106. In some embodiments in accordance with FIG. 7D, material 106 does not abut the spacer 140/material 110 stack. Rather, extended portion 193 of spacer 140 separates the spacer 140/material 110 stack from material 106 as illustrated in FIG. 7D. Extended portion 193 of spacer 140 provides the advantage of further separating material 106 and material 110 in order to prevent a short circuit between the two materials.

Referring to FIG. 7D, one embodiment of the present invention provides a biosensor including a plurality of devices 144 on a substrate 102. Each device 144 in the plurality of devices 144 occupies a different region on an insulator layer 104 and each device 144 in the plurality of devices 144 is capable of binding to a macromolecule 120. Insulator layer 104 is overlaid on substrate 102. Each device 144 in the plurality of devices 144 comprises (i) an electrically conducting material 106, (ii) a spacer 144 overlaid on a second portion of the different region of the insulator layer 104 that is occupied by the device 144, the spacer 144 including a main body and an extended portion 193, wherein extended portion 193 of spacer 144 abuts electrically conducting material 106, (iii) an electrically conducting material 110 that is overlaid on the main body of spacer 140, and (iv) a first passivation layer 130 that overlays the main body of spacer 140. In this embodiment, electrically conducting material 106 is overlaid on a first portion of the different region of the insulator layer that is occupied by the device.

Reference will now be made to FIG. 7E, which illustrates another biosensor in accordance with an embodiment of the present invention. The biosensor illustrated in FIG. 7E includes a substrate 102. Insulator layer 104 is overlaid on substrate 102. Insulator layer 104 is patterned to include steps 104-1 through 104-N. Steps 104-1 through 104-N are illustrated in FIG. 7E.

In one embodiment in accordance with FIG. 7E, a composition is deposited on each step 104-X of insulator 104 to form materials 106 through material 106-N. In this embodiment, a first pool of macromolecules 120 bridge material 106-1 and material 106-2, a second pool of macromolecules 120 bridge material 106-3 and material 106-4, and so forth, where each pool of macromolecules 120 is the same or different.

In other embodiments in accordance with FIG. 7E, steps in the set of steps 104-1 through 104-N are alternatively overlaid with materials 106 and 110 (not shown). For example, in one nonlimiting embodiment of the present invention, step 104-1 (FIG. 7E) is overlaid with material 106-1, step 104-2 is overlaid with material 110-1, step 104-3 is overlaid with material 106-2, step 104-4 is overlaid with material 110-2, and so forth. In this embodiment, a first pool of macromolecules 120 may bridge material 106-1 and material 110-1, a second pool of macromolecules 120 may bridge material 106-2 and material 110-2, and so forth, where each pool of macromolecules 120 is the same or different.

Referring to FIG. 7E, one embodiment of the present invention provides a biosensor. The biosensor comprises a substrate 102 and an insulator layer 104 overlaid on substrate 102. In the biosensor, the insulator layer 104 comprises a plurality of steps 104-X and a first step in the plurality of steps is at a different height, with respect to substrate 102, than a second step in the plurality of steps. Furthermore, each step in the plurality of steps is associated with a different electrically conducting layer 106 that is overlaid on the step. Each electrically conducting layer 106 on each step in the plurality of steps is electrically insulated from all other electrically conducting layers in the biosensor by insulator layer 104. In some embodiments, each electrically conducting layer 106 in the biosensor is addressable by an electrical source. For example, a voltage or electrical current may be applied to any desired electrically conducting layer 106 in the biosensor.

In some embodiments of biosensors in accordance with FIG. 7E, the difference in height, with respect to substrate 102, between a first step in the plurality of steps and a second step in the plurality of steps is in a range of 10 Angstroms to 30 Angstroms. In some embodiments of biosensors in accordance with FIG. 7E, the difference in height, with respect to substrate 102, between a first step in the plurality of steps and a second step in the plurality of steps is in a range of 25 Angstroms to 50 Angstroms. In some embodiments of biosensors in accordance with FIG. 7E, the difference in height, with respect to substrate 102, between a first step in the plurality of steps and a second step in the plurality of steps is in a range of 40 Angstroms to 80 Angstroms.

In some embodiments of the present invention, each step 104-N has a height 8902 (FIG. 7E) of between 5 and 10 Angstroms, between 10 and 15 Angstroms, between 15 and 20 Angstroms, between 20 and 25 Angstroms, between 25 and 30 Angstroms, between 35 and 40 Angstroms, between 40 Angstroms and 45 Angstroms, between 45 Angstroms and 50 Angstroms, between 50 Angstroms and 55 Angstroms, between 55 Angstroms and 60 Angstroms, between 60 Angstroms and 65 Angstroms, between 65 Angstroms and 70 Angstroms, between 70 Angstroms and 85 Angstroms, between 85 Angstroms and 100 Angstroms, or more than 100 Angstroms.

Referring to FIG. 7E, some embodiments of the present invention provide a biosensor having a plurality of steps in which a first step and a second step are adjacent to each other. Furthermore, a first portion of a macromolecule binds to the first step in the plurality of steps and a second portion of the macromolecule binds to the second step.

FIG. 7F shows a biosensor in accordance with another embodiment of the present invention. The biosensor illustrated in FIG. 7F comprises a substrate 102, a first insulator layer 104 overlaid on substrate 102, a first electrically conducting material 110 overlaid on insulator 104, and a passivation layer 130 overlaid on the first electrically conducting material 110. A crevice 9204 extends through the passivation layer 130, material 110, and first insulator layer 104. A second insulator layer 9208 in the crevice 9204 is found at the bottom of crevice 9204. Material 106 overlays second insulator layer 9208. In some embodiments, first insulator layer 104 has a thickness of between 10 Angstroms and 1500 Angstroms. In some embodiments, first insulator layer 104 has a thickness of between 250 Angstroms and 350 Angstroms. In yet other embodiments, first insulator layer 104 has a thickness of about 300 Angstroms and comprise silicon oxide. In still other embodiments, the first insulator layer 104 has a thickness between 700 Angstroms and 1300 Angstroms.

6.6 Biosensor Arrays

In various embodiments, there can exist multiple macromolecules 120 spanning a single pair of electrodes (e.g., a pair of materials 106 and 110) in a device 144 in accordance with the methods disclosed in Sections 6.1 through 6.4. Furthermore, there can be a multiplicity of electrode pairs (e.g., a multiplicity of devices 144) where each electrode pair is spanned by one or more macromolecules 120. Because of the small size of devices 144, a large number of devices 144 can be placed in a relatively small area (e.g. on a chip) thereby increasing sensitivity and improving signal to noise (S/N) ratio. In addition, assays can be performed using small quantities of sample.

A single substrate/chip can incorporate a number of different devices 144 thereby facilitating detection/quantification of a number of different analytes. Accordingly, in some embodiments, the biosensors of the present invention are arranged into arrays. Each array includes N devices 144. In practice, N is any number. In some embodiments, the biosensors of the present invention each comprise at least one device 144, at least two devices 144, at least ten devices 144, at least 100 devices 144, 1000 to 10,000 devices 144, 10,000 to 10⁵ devices 144, 10⁵ devices to 10⁷ devices 144, 10⁷ devices to 10⁹ devices 144, 10⁹ devices to 10¹¹ devices 144, 10¹¹ devices to 10¹² devices 144, or more. In some embodiments, each device 144 on a biosensor of the present invention is the same. In some embodiments, materials 106 and 110 are electrodes and each device 144 has an electrode-insulator-electrode configuration. In some embodiments at least two devices 144 in the biosensors of the present invention is different. It will be appreciated that each device 144 in the biosensors of the present invention may serve as an independent sensor for a particular application. Thus, in certain embodiments, an array of devices 144 on a single substrate 102 (e.g., chip) can detect/quantify two or more different analytes, four or more different analytes, 10 or more different analytes, 100 or more different analytes, 1000 or more different analytes, 10,000 more different analytes, 100,000 or more different analytes, or 1,000,000 or more different analytes.

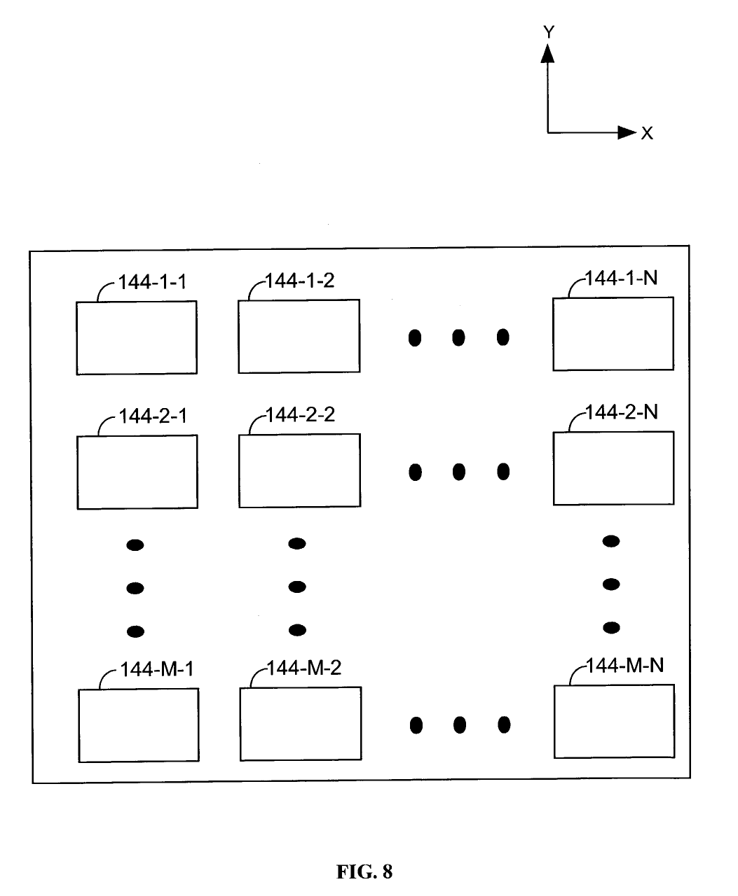

In some embodiments, the biosensors of the present invention comprise an array of discrete devices 144. An illustrative array of discrete devices in a biosensor of the present invention (e.g., biosensor 100, biosensor 200, biosensor 300, a biosensor illustrated in FIG. 7A-7E) is illustrated in FIG. 8. In the illustrative array shown in FIG. 8, there are N columns and M rows of devices 144. In some embodiments, N and M may be the same or a different number. In some embodiments, N and/or M has a value that is at least two, at least ten, at least 100, 1000 to 10,000, 10,000 to 10⁵, 10⁵ to 10⁷, 10⁷ to 10⁹, 10⁹ to 10¹¹, 10¹¹ to 10¹² devices, or more.

In some embodiments, the biosensors of the present invention include a plurality of devices 144 that are organized into one or more arrays. Each such array may have the configuration shown in FIG. 8, with N columns and M rows, where N and M may be the same or a different number. In some embodiments, the biosensors of the present invention include at least two arrays of devices 144, at least 10 arrays of devices 144, at least 100 arrays of devices 144, or at least 10² to 10²⁰ arrays of devices 144.

In some embodiments, each device 144 in a biosensor of the present invention is overlaid on an optional insulator layer 104. Optional insulator layer 104 is overlaid on substrate 102 in biosensors of the present invention. In some embodiments, there is no optional insulator layer 104 present in all or a portion of biosensors of the present invention and devices 144 are overlaid directly onto substrate 102.

The devices 144 in the biosensors of the present invention can adopt a wide variety of configurations. Thus, for example, in some embodiments of the present invention a macromolecule 120 does not span to material 106 and 110 in an electrode pair. Rather, a first macromolecule 120 is attached to material 106 and a second macromolecule 120 is attached to material 110. Binding of the analyte to the two macromolecules 120 can form an electrically conductive moiety that spans the gap between the two electrodes thereby allowing current to flow between the electrodes. Detection and measurement of this current allow for the detection/quantification of the bound analyte. Thus, for example, in one embodiment, the first and second macromolecules 120 are each nucleic acids complementary to half of the target analyte. When the analyte contacts the first and second macromolecules 120 under conditions permitting hybridization, the two macromolecules hybridize to the analyte forming a double-stranded nucleic acid spanning materials 106 and 110. The use of a first and second macromolecules 120 in this way can be performed with any of the biosensors described herein.

In another embodiment of the present invention, macromolecule 120 is attached to a material 106 of a device 144. The target analyte is tagged with a binding agent that causes the analyte to interact with and/or bind material 110. In use, the analyte binds to, e.g. material 110 and is bound by macromolecule 120. Together, the target analyte, with its binding agent, and macromolecule 120 bridge the gap between the electrodes 106 and 110 resulting in a detectable change in conductance. The use of a macromolecule 120 and a target analyte in this way can be performed with any of the biosensors described herein.

While a single macromolecule can span an electrode pair in a device 144, typically, a plurality of macromolecules 120 span any given electrode-pair in devices 144. Thus, in some embodiments, between two and ten, between ten and fifty, between fifty and 100, between 100 and 1,000, between 1,000 and 10,000, between 10,000 and 100,0000, or at least 1,000,000 macromolecules 120 span an electrode or electrode pair in a device 144 in a biosensor of the present invention.

6.7 Packaged Biosensor Arrays

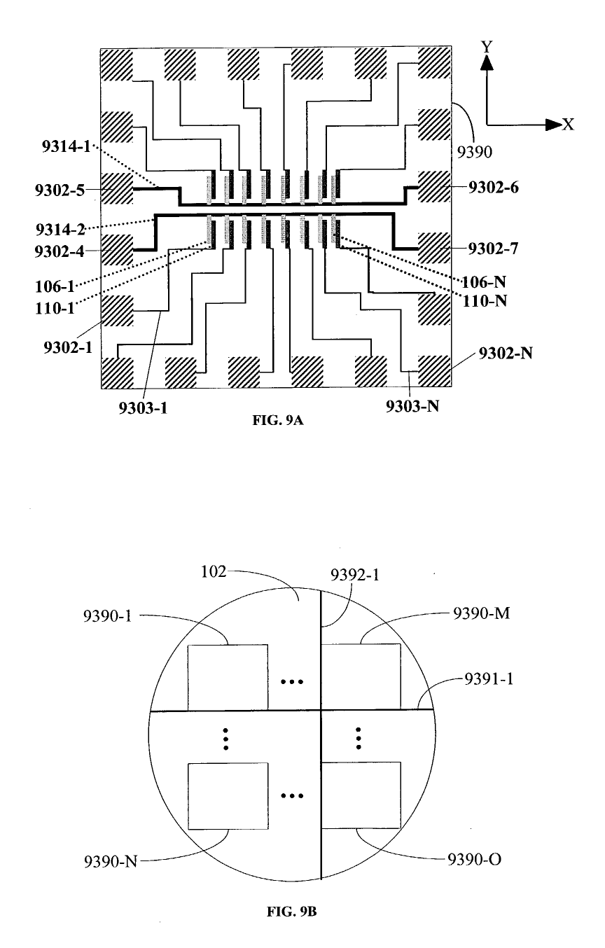

In some embodiments of the present invention, N devices 144 are arrayed on a substrate 102 that includes a plurality of upper steps 9310 and a plurality of lower steps 9308 (FIG. 9A). Each upper step 9310 in the plurality of upper steps is associated with a lower step 9308 in the plurality of lower steps. In various embodiments of the present invention, each upper step 9310 and associated lower step 9308 is separated in the Z-dimension (vertical dimension, i.e., perpendicular to the X-Y plane drawn in FIG. 9A) by 5 Angstroms to 100 Angstroms, 20 Angstroms to 80 Angstroms, 30 Angstroms to 60 Angstroms, about 40 Angstroms, about 50 Angstroms, about 75 Angstroms, about 80 Angstroms, about 100 Angstroms, about 125 Angstroms, about 150 Angstroms, about 200 Angstroms, more than 10 Angstroms, more than 40 Angstroms, more than 75 Angstroms, less than 200 Angstroms, or less than 100 Angstroms. In practice, the number N of devices 144 arrayed on the substrate illustrated in FIG. 9A is any number. In some embodiments, N is 1, 2, 10, at least 100, 1000 to 10,000, 10,000 to 10⁵, 10⁵ to 10⁷, 10⁷ to 10⁹, 10⁹ to 10¹¹, 10¹¹ to 10¹², or more.

In some embodiments of the present invention, material 110 of each device 144 is overlaid or integrated into upper step 9310 of substrate 102 and material 106 of each device 144 is overlaid or integrated into lower step 9308 of substrate 102 as illustrated in FIG. 9A. In some embodiments, substrate 102 illustrated (FIG. 9A) is patterned so that lower step 9308 includes elements 9320. Each element 9320 is bordered by portions of upper step 9310 as illustrated in FIG. 9A. In some embodiments of the present invention, each element 9320 has a width 9304 of between two and forty microns. In still other embodiments of the present invention, each element 9320 has a width 9304 of between four and thirty microns. In yet other embodiments of the present invention, each element 9320 has a width 9304 of between eight and twenty microns. In one embodiment of the present invention, each element 9320 has a width of about fifteen microns.

In some embodiments, the spacing 9302 between materials 110 of neighboring devices 144 overlaid on the substrate 102, as illustrated in FIG. 9A, is between 3 and 100 microns, between 5 and 80 microns, between 8 and 70 microns, between 10 and 50 microns, or between 15 and 40 microns. In some embodiments, the spacing 9302 between the material 110 of each neighboring device 144 overlaid on the substrate 102, as illustrated in FIG. 9A, is between 15 and 25 microns. In one embodiment, the spacing 9302 between the material 110 of each neighboring device 144 overlaid on the substrate 102 as illustrated in FIG. 9A is about twenty microns.

In some embodiments, the spacing 9306 between the material 106 of each neighboring device 144 overlaid on the substrate 102 as illustrated in FIG. 9A is between 3 and 80 microns, between 5 and 70 microns, between 7 and 60 microns, between 9 and 45 microns, or between 10 and 20 microns. In some embodiments, the spacing 9306 between materials 106 of each neighboring device 144 overlaid on the substrate 102 as illustrated in FIG. 9A is about fifteen microns.

Referring again to FIG. 9A, one aspect of the present invention provides a biosensor comprising a plurality of devices 144 on a substrate 102. Each device 144 in the plurality of devices 144 is for binding a macromolecule 120. In this aspect of the invention, substrate 102 comprises a plurality of upper steps 9310 and a plurality of lower steps 9308. Each upper step 9310 in the plurality of upper steps 9310 is associated with a lower step 9308 in the plurality of lower steps. An upper step 9310 is associated with a lower step 9308 when the two steps are adjacent to each other and a first electrically conducting material 110 overlays upper step 9310 and a corresponding second electrically conducting material 106 overlays the associated lower step 9308. A first electrically conducting material 106 and a second electrically conducting material 110 correspond to each other when they are within the same device 144. Thus, for each device 144 in the plurality of devices in the biosensor, a first electrically conducting material 110 in device 144 overlays an upper step 9310 in the plurality of upper steps and a second electrically conducting material 106 in device 144 overlays the lower step 9308, in the plurality of lower steps, that is associated with the upper step 9310.

6.7 Processing Steps used to Package Devices

In some embodiments of the present invention, electrode pairs 106-N/110-N are connected to external circuitry. FIG. 9A illustrates one such embodiment. Region 9390 (FIG. 9A) is referred to as a die. It will be appreciated that a large number of die (regions 9390) may be arranged on the same substrate 102. Thus, in some embodiments, many identical copies of die 9390 are manufactured simultaneously on a common substrate 102 (e.g. a wafer), as illustrated in FIG. 9B. In some embodiments of the present invention, there is ten or more die on a substrate (wafer) 102. In some embodiments of the present invention, there is 100 or more die on a substrate 102. In still other embodiment of the present is 1000 or more die on a substrate 102, 10000 or more die on a substrate 102, 100,000 or more die on a substrate 102, or 1,000,000 or more die on a substrate 102. Furthermore, each die (region 9390) may have any number of devices 144 connected to bonding pads 9302 using interconnect 9303. In some embodiments, there are 10 or more devices 144, 100 or more devices 144, 1000 or more devices 144, 10000 or more devices 144, 100,000 or more devices 144, or 1,000,000 or more device 144 in a given die. In some embodiments, there are between 100 and 1000 devices 144 on a substrate 102, between 1000 and 10,000 devices 144 on a substrate 102, or between 10,000 devices 144 and 100,000 devices 144 on a substrate.

In some embodiments of the present invention, each device 144 may not be directly connected to a bonding pad 9302 through an interconnect 9303. Rather, conventional circuit elements may be employed to selectively connect one device 144 to a given bonding pad 9302 via an address signal supplied via another one or more of bonding pads 9302. One circuit element that can be used for to accomplish this wiring scheme is a demultiplexer. A demultiplexer, having a complementary metal-oxide semiconductor (CMOS) architecture, can easily be incorporated into the biosensors of the present invention using semiconductor manufacturing techniques. See, also, Horowitz and Hill, The Art of Electronics, 2^{nd} edition, Cambridge University Press, 1989 at pp. 143-144. As will described in more detail below, each bonding pad 9302 is wired to a corresponding pin in a chip package. However, the number of pins in a package is limited. Therefore, the use of circuit elements, such as demultiplexers, is advantageous because it allows for chip configurations in which the number of devices 144 in the chip is much larger than the number of pins in the chip.

After patterning and metallization, it is desired to place a particular die 9390 into a package (a process referred to as packaging) to protect interconnects 9303 on the die from liquids used to expose devices 144 to macromolecules 120 and/or analytes used to bind to macromolecules 120. Furthermore, the packaging facilitates connection of the die with a printed circuit (PC) board (not shown). A printed circuit (PC) board is a rigid, insulating sheet of material with thin plated cooper lines forming circuit paths used to facilitate connection of devices 144 on die 9390 to external circuitry. As such, a printed circuit board contains the logic used to electronically address each device 144 in die 9390. In alternative embodiments in accordance with the present invention, die 9390 is part of a multi-chip module (MCM) or is integrated directly with the external circuitry.

The first step in a packaging process in accordance with one embodiment of the present invention is to separate a selected die 9390 (FIG. 9B) from wafer (substrate) 102. One possible method of achieving this is to use sawing. In sawing, a diamond-impregnated saw is first passed over first-oriented scribe lines (e.g., FIG. 9B, 9391-1) on the wafer and then second-oriented scribe lines (e.g., FIG. 9B, 9392-1) on the wafer. In one version of the sawing method, the saw cuts completely through the thickness of the wafer 102, separating a die 9390. Alternatively, the saw is used to make trenches of some depth in the wafer 102 (substrate), collocated with the scribe lines. Subsequently, the wafer is mechanically stressed by moving a roller over the surface, separating die 9390 from wafer 102.

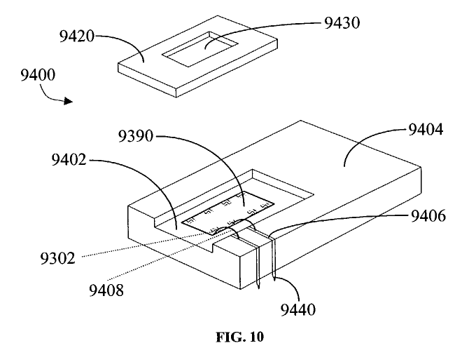

After a die 9390 has been separated from wafer 102, it is placed in a package. As depicted in FIG. 10, in one embodiment die 9390 is placed on die-attach surface 9402 of package body 9404. To create a strong mechanical coupling between the die 9390 and package body 9404, a die-attachment bond is formed between the underside of the die 9402 and the die-attach surface 9402. In one possible embodiment, a thick liquid epoxy adhesive such as silver filled epoxy is used to achieve the desired bond. In this embodiment, before die 9390 is placed on die-attach surface 9402, the epoxy is deposited on the die-attach surface 9402. Then, die 9390 is placed onto the die-attach surface 9402, forcing the epoxy to form a thin layer of uniform thickness. Finally, entire assembly 9400 is placed into a curing oven and heated. The elevated temperature induced by the curing oven causes the epoxy to create a permanent bond between die 9390 and die-attach surface 9402.

In another embodiment, die 9390 is attached to die-attach surface 9402 by cutectic die attachment. The eutetic method is named for the phenomenon that takes place when two materials melt together (alloy) at a much lower temperature than either of them separately. For die attach, two eutectic materials are gold and silicon. On one eutetic method, gold is plated on die attach surface 9402. Then, when die 9390 is pressed onto surface 9402, the gold alloys with the silicon substrate upon heating. See, for example, Van Zant, Microchip Fabrication: A Practical Guide to Semiconductor Processing, 4^{th} edition, McGraw-Hill, 2000 at p. 570.

After die attachment, the next step in the packaging process is to attach each die bonding pad 9302 (see also FIG. 9A) to each package lead via 9406 with bond wires 9408. In one embodiment of the present invention, this step is performed using wire bonding. Alternatively, flip-chip or beam-lead techniques can be used. See, for example, Streetman, Solid State Electronic Devices, 4^{th} Edition, Prentice Hall (NJ), 1995 at p. 371. In wire bonding, bond wire 9408 is a thin (0.7-1.0 mil) wire, sometimes composed of gold (Au) or aluminum (Al). The process of wire bonding begins by placing bond wire 9408 into capillary device. The capillary device is then positioned such that the end of bond wire 9408 is positioned directly over die bonding pad 9302. A combination of downward mechanical pressure applied by the capillary device and heat (as in thermocompression bonding) or ultrasonic energy (as in thermosonic bonding) then causes the end of bonding wire 9408 to form a strong metallic bond between bond wire 9408 and bonding pad 9302. Next, the capillary device is positioned such that a portion of bond wire 9408 is positioned directly over package lead 9406. Again, a combination of pressure and heat or ultrasonication serves to form a metallic bond between bond wire 9408 and package lead 9406. See, for example, Streetman, Solid State Electronic Devices, 4^{th} Edition, Prentice Hall (NJ), 1995 at pp. 368-370. Subsequently, the process is repeated with a new bonding wire 9408, which is bonded to a different contact pad 9302 and package lead 9406. This process is repeated until all of the contact pads 9302 of the die 9390 that are desired to be connected to external circuitry are wire bonded by a separate bond wire 9408 to a separate package lead 9406.

FIG. 10 illustrates a dual in-line package (DIP), so-named for the two linear series of pins 9440 (only one linear series is shown). In other embodiments of the present invention, other package types are used. Such package types include, but are not limited to, single in-line package (SIP) or ball grid array (BGA) packages. See also Van Zant, Microchip Fabrication: A Practical Guide to Semiconductor Processing, 4^{th} edition, McGraw-Hill, 2000, at p. 570.

In a DIP package, die 9390 is enclosed in a ceramic or plastic case for mechanical, thermal, and electrical protection of the die. In one embodiment of the present invention, the last step in the packaging process is to fully enclose die 9390 by placing upper piece 9420 onto the die attach surface of package body 9404. In one embodiment, package body 9404 and upper piece 9420 are composed of a ceramic. Epoxy is deposited on the die-attach surface 9402. Then, upper piece 9420 is placed onto die-attach surface 9402, forcing the epoxy to form a thin layer of uniform thickness. Finally, the entire assembly 9400 is placed into a curing oven and heated. The elevated temperature induced by the oven causes the epoxy to create a permanent bond between upper piece 9420 and package body 9404. In some embodiments of the present invention, upper piece 9420 has access hole 9430 so that, after sealing to package body 9404, it is still possible to access the active area of die 9390. The active area of die 9390 is that portion of die 9390 that has one or more devices 144. Access hole 9430 is used, for example, to expose devices 144 to macromolecules 120, analytes, or rinse solutions.

6.8 Interfacing Packaged Devices

One aspect of the present invention provides methods for interfacing a biosensor with data acquisition and signal generation equipment. Such an interface allows for automated measurement of a current through a device 144 in the biosensor when a voltage is applied across device 140. Other electrical properties, including but not limited to capacitance, inductance, and resistance, of a device 144 may also be determined. The voltage applied to a device 144 may be a direct current (DC) voltage, an alternating current (AC) voltage of a given frequency, or an arbitrary waveform, such as sawtooth. This aspect of the present invention allows for the automated measurement of the current response of each individual device 144 to application of such a voltage.

In one embodiment in accordance with this aspect of the invention, the automated system incorporates a computer having a microprocessor. This computer can have any of a wide range of architectures such as, for example the personal computer (PC) architecture. Software stored on computer is used to automatically measure the properties of devices 144 that are of interest and to store the results for either subsequent or immediate interpretation.

To facilitate connection of the devices 144 in a packaged biosensor with a computer, package 9404 is attached to a printed circuit board. A printed circuit board is a piece of rigid insulating material with holes for the insertion of package pins 9440 and thin plated copper lines for forming the circuit paths between the pins 9440 and devices external to the printed circuit board. In one embodiment, each of the package pins 9440 is connected by a copper line on the printed circuit board to a corresponding lead of an edge connector. The edge connector itself can mate directly with other connectors in a variety of industry standard ways. Other methods may also be used to connect the packaged biosensor to devices external to the board. See, for example, Horowitz and Hill, The Art of Electronics, 2^{nd} edition, Cambridge University Press, 1989 at pp. 837-838.

The printed circuit boards may then be electrically interfaced to the computer. Any one of a number of commercially available data acquisition cards (DAC) can be used for this purpose. For example, part ADAC/5503HR (IOtech, Inc. Cleveland, Ohio) provides a number of user-programmable analog output channels. These output channels could be used to apply a voltage across the electrodes in a device 144. To measure the current that results, a digital multimeter such as the 34401A Digital Multimeter (Agilent Technologies, Palo Alto, Calif.) can be connected in series in the circuit loop formed by the output channel of the DAC card and the device 144. The 34401A digital multimeter can be interfaced to the computer using, for example a standard RS232 serial interface. The DAC ADAC/5503HR can be interfaced to the PC using, for example, a PCI (Peripheral Component Interconnect) bus card slot in the computer

In addition to the hardware described above that is used for coupling the computer to the device 144, it is advantageous to provide software instruction to the computer as to how to automatically apply voltages across specific device pair 144 and measure the result. In one embodiment, LabView© (National Instruments Corporation, Austin, Tex.) is used to provide a high-level computer programming language that facilitates the development of computer software for this purpose. The software can be adapted to instruct the DAC to apply a voltage to a specific device 144 in the packaged biosensor by applying a voltage to the corresponding channel of the output of the DAC. Then, the software can request that the current measured by the digital multimeter be acquired and stored in the memory or hard drive of the computer. Furthermore, IntuiLink© software (Agilent Technologies, Palo Alto, Calif.) can be used to facilitate communication between the multimeter and the computer. One of skill in the art will recognize that any one of a number of high-level computer programming languages can be used for this purpose (C, C++, Perl, Fortran, Visual Basic, etc.). The process of applying a voltage to specific device 144 in the package biosensor and measuring the resulting current can then be repeated for each desired device 144 in package 9404 in any manner desired.

6.9 Substrates used in the Biosensors of the Present Invention

In some embodiments, substrate 102 is nonconductive. In some embodiments, substrate 102 is an insulator. In some embodiments, substrate 102 is made of a material such as silicon, silicon oxide, silicon dioxide, silicon nitride, Teflon, or alumina. In some embodiments, substrate 102 is made of glass. For example, in some embodiments, substrate 102 is made from a 600 cm×800 cm motherglass, a 1 meter×1.2-meter motherglass, or larger. In some embodiments of the present invention substrate 102 is made of polyester. In some embodiments, substrate 102 is made out of sapphire, nitrides, arsenides, carbides, oxides, phosphides, or selinides. In some embodiments, substrate 102 is Alkali-free borosilicate glass (Shott AF45).

6.10 Composition of Biosensor Electrodes

In some embodiments, materials 106 and 110 serve as electrodes in biosensors of the present invention. Accordingly, in some embodiments of the present invention, materials 106 and 110 are formed from essentially any conductive material. For example, in some embodiments of the present invention, material 106 and/or material 110 has a resistivity of less than 10⁻³ ohm-meters, less than 10⁻⁴ ohm meters, less than 10⁻⁶ ohm meters, or less than 10⁻⁷ ohm meters.

In some embodiments of the present invention, materials 106 and 110 are made of the same composition. In other embodiments of the present invention, materials 106 and 110 are made of different compositions. In some embodiments, material 106 and/or material 110 comprise silicon, dense silicon carbide, boron carbide, Fe₃O₄, germanium, silicone germanium, silicon carbide, polysilicon, tungsten carbide, titanium carbide, indium phosphide, gallium nitride, gallium phosphide, aluminum phosphide, aluminum arsenide, mercury cadmium telluride, tellurium, selenium, ZnS, ZnO, ZnSe, CdS, ZnTe, GaSe, CdSe, CdTe, GaAs, InP, GaSb, InAs, Te, PbS, InSb, InSb, PbTe, PbSe, tungsten disulfide, or any combination thereof.

In some embodiments, material 106 and/or material 110 comprises a metal. In some embodiments, material 106 and/or material 110 is made of a material selected from the group consisting of ruthenium, cobalt, rhodium, rubidium, lithium, sodium potassium, vanadium, cesium, chromium, molybdenum, silicon, germanium, aluminum, iridium, nickel, palladium, platinum, iron, copper, titanium, tungsten, silver, gold, zinc, cadmium, indium tin oxide, carbon, carbon nanotube, or alloys or compounds of such materials.

In some embodiments, material 106 and/or material 110 comprises a metal carbide, metal nitride, or metal boride (e.g., tungsten, titanium, iron, niobium, vanadium, zirconium, hafnium, molybdenum, etc.). In some embodiments, material 106 and/or material 110 comprises a conductive oxide (e.g., transition element monoxide, dioxides and sequioxides, perovskite and perovskite related oxides such as strontinates and lanthanates). In some embodiments, material 106 and/or material 110 comprises a metal silicide or a metal sulfide. In some embodiments, material 106 and/or material 110 comprises a semiconductor or compound semiconductor material.

6.11 Composition of Biosensor Insulators

In some embodiments of the present invention spacer 140 and optional insulator 104 are made from the same materials. In other embodiments of the present invention, spacer 140 and optional insulator 104 are made from different materials. In some embodiments, materials used to make insulator 104 and/or spacer 140 include elements, compounds and substances that have a resistivity greater than about 10⁻³ ohm-meters. In some embodiments, materials used to make insulator 104 and/or spacer 140 include elements, compounds and substances that have a resistivity greater than about 10⁻² ohm-meters, greater than about 10⁻¹ ohm-meters, or greater than about 10 ohm-meters. In some embodiments of the present invention insulator 104 and/or spacer 140 is made of high resistivity plastic. A “high resistivity plastic” refers to a plastic with a resistivity greater than 10⁻³ ohm-meters, greater than 10⁻² ohm-meters, greater than 10⁻¹ ohm-meters, greater than 1 ohm-meter, or greater than 10 ohm-meters.

In some embodiments, spacer 140 and/or insulator 104 is made from a material such as TiO, ZrO₂, Al₂O₃, CaF₂, Cr₂O₃, Er₂O₃, HfO₂, MgF₂, MgO, Si₃N₄, SnO₂, quarts, porcelain, tantalum pentoxide, silicon oxide, silicon nitride, ceramic, polystyrene, Teflon, insulating carbon derivatives, glass, clay, polystyrene, or an insulating oxide or sulfide of a transition metal in the periodic table of elements. The transition metals comprise groups IIIB, IVB, VB, VIIB, VIIIB, IB, and IIB of the periodic table. This group of elements is defined herein as the d-block. In addition to the d-block, transition metals comprise lanthanides and main group elements having chemical properties similar to transition metals. As defined herein, lanthanides are the first row of the f-block of the periodic table and main group elements are those in groups IIIA, IVA, VA and VIIA of the periodic table, the first five groups of which is known to those of skill in the art as the p-block. (See, e.g., Huheey, Inorganic Chemistry, Harper & Row, New York, 1983).

In some embodiments, spacer 140 and/or insulator 104 comprises an air gap insulator, a stoichiometric oxide, a stoichiometric nitride, an off stoichiometric oxide, an off stoichiometric nitride, a polymeric film (e.g., polystyrene or Teflon), an insulting carbon, or an insulating sulfide.

In some embodiments, spacer 140 and/or insulator 104 comprises porcelain, Teflon, ceramics, polymers, or rubber. In some embodiments, where possible, spacer 140 and/or insulator 104 comprises dry air. In some specific embodiments, spacer 140 and/or insulator 104 comprises SiO₂, Al₂O₃, Fe₂O₃, MgO, SrTiO₃, MgAl₂O₄, YBa₂Cu₃O₇₋ₓ, Si₃N₄, TiN, AlN, GaN, BN, SiC, WC, or TiC. In still other embodiments, spacer 140 and/or insulator 104 comprises SiO₂, fluorinated silicate glass, polycrystalline diamond films, or diamond-like carbon (DLC),

6.12 Composition of Biosensor Passivation Layer

In one embodiment of the present invention, passivation layer 130 is any material that does not bind to sulfur. In another embodiment of the present invention, passivation layer 130 is a layer that does not bind to macromolecules 120. In some embodiments of the present invention, passivation layer 130 is a material such as silicon oxide, silicon dioxide, silicon nitride, or silicon oxy-nitride. In some embodiments of the present invention, passivation layer 130 is an organic film such as polyamide. In yet other embodiments of the present invention, passivation layer 130 comprises aluminum having a thin layer of oxidation (oxidized aluminum). The thin layer of oxidation prevents sulfur binding. In some embodiments of the present invention, macromolecule 120 includes sulfur-based groups (e.g., thiols, sulfides) that bind to materials 106 and 110 on the biosensors of the present invention thereby bridging materials 106 and 110.

6.13 Composition of Target Biological Macromolecules

In some embodiments, macromolecule 120 is a biological molecule such as a polymer (e.g., nucleic acid, protein, polypeptide, peptide, antibody), carbohydrate, polysaccharide, lipid, fatty acid, sugar, and the like. Macromolecules 120 include, but are not limited to, receptors, ligands for receptors, antibodies or binding portions thereof (e.g., Fab, (Fab)′₂), proteins or fragments thereof, nucleic acids, oligonucleotides, glycoproteins, polysaccharides, antigens, epitopes, carbohydrate moieties, enzymes, enzyme substrates, lectins, protein A, protein G, organic compounds, organometallic compounds, lipids, fatty acids, lipopolysaccharides, peptides, cellular metabolites, hormones, pharmacological agents, tranquilizers, barbiturates, alkaloids, steroids, vitamins, amino acids, sugars, nonbiological polymers, biotin, avidin, streptavidin, organic linking compounds such as polymer resins, lipoproteins, cytokines, lymphokines, hormones, synthetic polymers, organic and inorganic molecules, etc.

The terms “polypeptide”, “peptide” and “protein” are used interchangeably herein to refer to a polymer of amino acid residues. Such polymers may include one or more amino acid residues that are an artificial chemical analogue of a corresponding naturally occurring amino. The term “nucleic acid” as used herein refers to a deoxyribonucleotide or ribonucleotide in either single- or double-stranded form. The term encompasses nucleic acids, e.g., oligonucleotides, containing known analogues of natural nucleotides that have similar or improved binding properties, for the purposes desired, as the reference nucleic acid. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompasses by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6:153-156). The term nucleic acid is used interchangeably with gene, cDNA, mRNA, oligonucleotide primer, probe and amplification product.

The term “antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Fundamental Immunology, Third Edition, W. E. Paul, ed., Raven Press, N.Y. 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies, those synthesized de novo using recombinant DNA methodologies (e.g., single chain Fv), and those found in display libraries (e.g. phage display libraries).

In one embodiment, macromolecule 120 is a single-stranded nucleic acid. In some embodiments in which macromolecule 120 is a single-stranded nucleic acid, the nucleic acid is derivatized at each terminus with a linker that physically and electrically couples the nucleic acid to respective materials 106 and 110 such that the nucleic acid spans the gap between the materials. Single-stranded nucleic acids are essentially nonconductive. However, when the nucleic acid binding agent is contacted with a complementary nucleic acid analyte under conditions that permit nucleic acid hybridization, the analyte nucleic acid binds to the sensor nucleic acid via complementary base pairing to form a double stranded hybrid duplex spanning the electrodes. This double stranded duplex is electrically conductive. The change in conductivity caused by such binding is readily detected using electrical/electrochemical means.

Macromolecule 120 is not limited to a nucleic acid. Any number of other macromolecules 120 can also be used in the biosensors of the present invention. Generally, macromolecules 120 are selected that are capable of specifically binding to a particular target analyte. Such macromolecules 120 include, but are not limited to, nucleic acids (including, but not limited to single stranded DNA or RNA, double stranded DNA or RNA, peptide nucleic acids, phosphorothioates, and the like), proteins, antibodies, lectins, sugars, lipids, polysaccharides, and the like.

In some embodiments, macromolecules 120 are utilized in the biosensors of the present invention that are not conductive in the absence of an analyte. These macromolecules 120 preferably form an electrically conductive complex when bound to an analyte. However, the biosensors of the present invention are not limited to macromolecules 120 that are not electrically conductive in the absence of an analyte. In certain embodiments it is sufficient that the analyte/macromolecule 120 complex exhibits a different electrical conductivity than the uncomplexed macromolecule 120.

Alternatively, where the analyte/macromolecule 120 complex shows the same conductivity as the uncomplexed macromolecule 120, it is possible to use various chemical agents that intercalate into the analyte/macromolecule 120 complex in order to change the effective conductivity of the complex. In some embodiments, an uncomplexed macromolecule 120 affords fewer intercalation sites relative to the analyte/macromolecule 120. Thus, the analyte/macromolecule 120 complex intercalates a greater number of intercalation agents relative to the uncomplexed macromolecule 120. Because of this, the analyte/macromolecule 120 exhibits a conductivity that is different than the conductivity exhibited by the uncomplexed macromolecule 120.

Intercalating reagents that change the conductivity of a macromolecule 120 or an analyte/macromolecule 120 complex are well known to those of skill in the art. Such intercalators include, but are not limited to, redox-active cations (e.g. Ru(NH₃)₆³⁺ and various transition metal/ligand complexes. Suitable transition metals for use in the invention include, but are not limited to, cadmium (Cd), magnesium (Mg), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metal, the platinum metals (Ru, Rh, Pd, Os, Ir and Pt), along with Re, W, Mo and Tc, are preferred. Particularly preferred are ruthenium, rhenium, osmium, platinum and iron.

In some embodiments, transition metals are complexed with a variety of ligands to form suitable transition metal complexes. Suitable ligands include, but are not limited to, amine, pyridine, pyrazine, isonicotinamide, imidazole, bipyridine, substituted derivatives of bipyridine, phenanthrolines (e.g., 1,10-phenanthroline), substituted derivatives of phenanthrolines (e.g., 4,7-dimethylphenanthroline), dipyridophenazine 1,4,5,8,9,12-hexaazatriphenylene, 9,10-phenanthrenequinone diimine, 1,4,5,8-tetraazaphenanthrene, 1,4,8,11-tetra-azacyclotetradecane, diaminopyridine; porphyrins and substituted derivatives of the porphyrin family.

Such intercalating reagents can also be used to detect mismatches between macromolecule 120 and the target analyte. Thus, for example, where macromolecule 120 and the analyte are nucleic acids, intercalating reagents comprising dimeric naphthyridines will specifically intercalate and localize where there is a G-G mismatch between the binding reagent and the target analyte (see, e.g., Nakatani et al., 2001, Nature/Biotechnology, 19: 51-55). Such mismatch specific reagents can be used to detect or screen for single nucleotide polymorphisms (SNPs).

6.14 Analytes used to Bind to Target Biological Macromolecules in the Present Invention

Analytes used to bind to macromolecules 120 include, but are not limited to, whole cells, subcellular particles, viruses, prions, viroids, nucleic acids, proteins, antigens, lipoproteins, lipopolysaccharides, lipids, glycoproteins, carbohydrate moieties, cellulose derivatives, antibodies, fragments of antibodies, peptides, hormones, pharmacological agents, cellular components, organic compounds, non-biological polymers, synthetic organic molecules, organo-metallic compounds, and inorganic molecules.

In some embodiments of the present invention, the analyte is purified. In some embodiments, the analyte is found in a sample. The sample can be derived from, for example, a solid, emulsion, suspension, liquid or gas. Furthermore, the sample may be derived from, for example, body fluids or tissues, water, food, blood, serum, plasma, urine, feces, tissue, saliva, oils, organic solvents, earth, water, air, or food products. The sample may comprise a reducing agent or an oxidizing agent, solubilizer, diluent, preservative, or other suitable agents.

Macromolecule 120 and its target analyte can exist as a pair of “binding partners”, e.g. a ligand and its cognate receptor, an antibody and its epitope, etc. Thus, a biological “binding partner” or a member of a “binding pair” refers to a molecule or composition that specifically binds other molecules to form a binding complex such as antibody-antigen, lectin-carbohydrate, nucleic acid-nucleic acid, biotin-avidin, etc.

The analytes used in this invention are selected based upon the characteristics of the macromolecules 120 that are to be identified/quantified. Thus, for example, where macromolecule 120 is a nucleic acid the analyte is preferably a nucleic acid or a nucleic acid binding protein. Where macromolecule 120 is a protein, the analyte is preferably a receptor, a ligand, or an antibody that specifically binds macromolecule 120. Where the macromolecule 120 is a sugar or glycoprotein, the analyte is preferably a lectin, and so forth.

6.15 Preparation of Macromolecules and Analytes

Methods of synthesizing or isolating suitable macromolecules 120 are well known to those of skill in the art as explained below.

6.15.1 Preparation of Macromolecules or Analytes that are Nucleic Acids

Nucleic acids for use as macromolecules 120 or analytes that bind to macromolecules 120 are produced or isolated according to any of a number of known methods. In one embodiment, the nucleic acid is an isolated naturally occurring nucleic acid (e.g., genomic DNA, cDNA, mRNA, etc.). Methods of isolating naturally occurring nucleic acids are known. See, for example, Sambrook et al., 1989, Molecular Cloning—A Laboratory Manua, 2^{nd} Ed., volumes 1-3, Cold Spring Harbor Laboratory, Cold Spring Harbor, N.Y.

In one embodiment, the nucleic acid is created de novo. In one example, the nucleic acid is created through chemical synthesis, e.g., according to the solid phase phosphoramidite triester method described by Beaucage and Caruthers, 1981, Tetrahedron Letts., 22(20): 1859-1862, e.g., using an automated synthesizer, as described in Needham-VanDcvanter et al, 1984, Nucleic Acids Res., 12: 6159-6168. Purification of oligonucleotides, where necessary, is typically performed by either native acrylamide gel electrophoresis or by anion-exchange HPLC as described in Pearson and Regnier, 1983, J. Chrom. 255: 137-149. The sequence of the synthetic oligonucleotides can be verified using the chemical degradation method of Maxam and Gilbert, 1980, in Grossman and Moldave (eds.) Academic Press, New York, Meth. Enzymol. 65: 499-560.

6.15.2 Preparation of Macromolecules or Analytes that are Antibodies or Antibody Fragments

Antibodies or antibody fragments for use as macromolecules 120 or as analytes that bind to macromolecules 120 can be produced by a number of methods well known to those of skill in the art. See, for example, Harlow & Lane, 1988, Antibodies: A Laboratory Manual, Cold Spring Harbor Laboratory, and Asai, 1993, Methods in Cell Biology Vol. 37: Antibodies in Cell Biology, Academic Press, Inc. N.Y. In one approach, antibodies are produced by immunizing an animal (e.g. a rabbit) with an immunogen containing a desired epitope. A number of immunogens may be used to produce specifically reactive antibodies. Recombinant protein is the preferred immunogen for the production of monoclonal or polyclonal antibodies. Naturally occurring proteins may also be used either in pure or impure form. Synthetic peptides can also be made using standard peptide synthesis chemistry (see, e.g., Barany and Merrifield, Solid-Phase Peptide Synthesis; pp.3-284 in The Peptides: Analysis, Synthesis, Biology. Vol. 2: Special Methods in Peptide Synthesis, Part A., Merrifield et al. (1963) J. Am. Chem. Soc., 85: 2149-2156, and Stewart et al. (1984) Solid Phase Peptide Synthesis, 2^{nd} ed. Pierce Chem. Co., Rockford, Ill.)

Methods of production of polyclonal antibodies are known to those of skill in the art. In brief, an immunogen is mixed with an adjuvant and animals are immunized. The animal's immune response to the immunogen preparation is monitored by taking test bleeds and determining the titer of reactivity to the immunogen. When appropriately high titers of antibody to the immunogen are obtained, blood is collected from the animal and antisera are prepared. Further fractionation of the antisera to enrich for antibodies reactive to the immunogen can be done if desired. See, for example, Harlow & Lane, 1988, Antibodies: A Laboratory Manual, Cold Spring Harbor Laboratory.

Monoclonal antibodies may be obtained by various techniques familiar to those skilled in the art. Briefly, spleen cells from an animal immunized with a desired antigen are immortalized, commonly by fusion with a myeloma cell. See, for example, Kohler and Milstein, 1976, Eur. J. Immunol. 6: 511-519. Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells according to the general protocol outlined by Huse et al., 1989, Science, 246:1275-1281.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment from a library of greater than 10¹⁰ nonbinding clones. To express antibody fragments on the surface of phage (phage display), an antibody fragment gene is inserted into the gene encoding a phage surface protein (pIII) and the antibody fragment-pIII fusion protein is displayed on the phage surface (McCafferty et al., 1990, Nature, 348: 552-554; Hoogenboom et al., 1991, Nucleic Acids Res. 19: 4133-4137).

Since the antibody fragments on the surface of the phage are functional, phage bearing antigen binding antibody fragments can be separated from non-binding phage by antigen affinity chromatography (McCafferty et al., 1990, Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al., 1990, Nature, 348: 552-554). Thus even when enrichments are low (Marks et al., 1991, J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (e.g., several hundred) need to be analyzed for binding to antigen.

Human antibodies can be produced without prior immunization by displaying very large and diverse V-gene repertoires on phage (Marks et al., 1991, J. Mol. Biol. 222: 581-597). In one embodiment, natural V_{H} and V_{L} repertoires present in human peripheral blood lymphocytes are isolated from unimmunized donors by PCR. The V-gene repertoires are spliced together at random using PCR to create a scFv gene repertoire that is cloned into a phage vector to create a library of 30 million phage antibodies (Id.). From this single “naive” phage antibody library, binding antibody fragments are isolated against different antigens, including haptens, polysaccharides and proteins (Marks et al (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Furthermore, antibodies can be produced against self-proteins, including human thyroglobulin, immunoglobulin, tumor necrosis factor and CEA (Griffiths et al., 1993, EMBO J. 12: 725-734). It is also possible to isolate antibodies against cell surface antigens by selecting directly on intact cells. The antibody fragments are highly specific for the antigen used for selection and have affinities in the 1 μM to 100 nM range (Marks et al., 1991, J. Mol. Biol. 222: 581-597; Griffiths et al. (1993) EMBO J. 12: 725-734). Larger phage antibody libraries result in the isolation of more antibodies of higher binding affinity to a greater proportion of antigens.

6.15.3 Preparation of Macromolecules or Analytes that are Proteins

Suitable proteins for use as macromolecules 120 or analytes include, but are not limited to, receptors (e.g. cell surface receptors), receptor ligands, cytokines, transcription factors and other nucleic acid binding proteins, growth factors, etc.

The protein can be isolated from natural sources, mutagenized from isolated proteins, or synthesized de novo. Means of isolating naturally occurring proteins are well known to those of skill in the art. Such methods include, but are not limited to, well known protein purification methods including ammonium sulfate precipitation, affinity columns, column chromatography, gel electrophoresis and the like (see, generally, R. Scopes, 1982, Protein Purification, Springer-Verlag, N.Y.; Deutscher, 1990, Methods in Enzymology Vol. 182: Guide to Protein Purification, Academic Press, Inc. N.Y.).

Where the protein binds a target reversibly, affinity columns bearing the target can be used to affinity purify the protein. Alternatively, the protein can be recombinantly expressed with a HIS-Tag and purified using Ni²⁺/NTA chromatography. In another embodiment, the protein can be chemically synthesized using standard chemical peptide synthesis techniques. Where the desired subsequences are relatively short the molecule may be synthesized as a single contiguous polypeptide. Where larger molecules are desired, subsequences can be synthesized separately (in one or more units) and then fused by condensation of the amino terminus of one molecule with the carboxyl terminus of the other molecule thereby forming a peptide bond. This is typically accomplished using the same chemistry (e.g., Fmoc, Tboc) used to couple single amino acids in commercial peptide synthesizers.

Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany and Merrifield (1962) Solid-Phase Peptide Synthesis; pp. 3-284 in The Peptides: Analysis, Synthesis, Biology. Vol. 2: Special Methods in Peptide Synthesis, Part A., Merrifield et al. (1963) J. Am. Chem. Soc., 85: 2149-2156, and Stewart et al. (1984) Solid Phase Peptide Synthesis, 2^{nd} ed. Pierce Chem. Co., Rockford, Ill.

In a preferred embodiment, the protein can also be synthesized using recombinant DNA methodology. Generally, this involves creating a DNA sequence that encodes the binding protein, placing the DNA in an expression cassette under the control of a particular promoter, expressing the protein in a host, isolating the expressed protein and, if required, renaturing the protein.

DNA encoding binding proteins or subsequences of this invention can be prepared by any suitable method as described above, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

The nucleic acid sequences encoding the desired binding protein(s) may be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli, this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes. Once expressed, the recombinant binding proteins can be purified according to standard procedures of the art as described above.

6.15.4 Preparation of Macromolecules or Analytes that are Sugars or Carbohydrates

Sugars and carbohydrates can be isolated from natural sources, enzymatically synthesized or chemically synthesized. A route to production of specific oligosaccharide structures is through the use of the enzymes that make them in vivo; the glycosyltransferases. Such enzymes can be used as regio- and stereoselective catalysts for the in vitro synthesis of oligosaccharides (Ichikawa et al., 1992, Anal. Biochem. 202: 215-238). Sialyltransferase can be used in combination with additional glycosyltransferases. For example, a combination of sialyltransferase and galactosyltransferases can be used. A number of methods of using glycosyltransferases to synthesize desired oligosaccharide structures are known. Exemplary methods are described, for instance, in WO 96/32491; Ito et al., 1993, Pure Appl. Chem. 65:753, and U.S. Pat. Nos. 5,352,670, 5,374,541, and 5,545,553. The enzymes and substrates can be combined in an initial reaction mixture, or alternatively, the enzymes and reagents for a second glycosyltransferase cycle can be added to the reaction medium once the first glycosyltransferase cycle has neared completion. By conducting two glycosyltransferase cycles in sequence in a single vessel, overall yields are improved over procedures in which an intermediate species is isolated.

Methods of chemical synthesis are described by Zhang et al., 1999, J. Am. Chem. Soc., 121(4): 734-753. Briefly, in this approach, a set of sugar-based building blocks is created with each block preloaded with different protecting groups. The building blocks are ranked by reactivity of each protecting group. A computer program then determines exactly which building blocks must be added to the reaction so that the sequences of reactions from fastest to slowest produces the desired compound.

6.16 Spacing Between Biosensor Electrodes

The spacing between materials 106 and 110 in the biosensors of the present invention is designed so that a macromolecule 120 can span materials 106 and 110 by binding to both materials 106 and 110. Accordingly, the spacing between materials 106 and 110 will depend upon the size of the macromolecules 120 analyzed. The spacing between the top of material 106 and the top of material 110 is illustrated in FIGS. 1, 2, and 3 as element 190. The spacing between materials 106 and 110 is further illustrated as element 121 in FIGS. 4, 5, and 6. In other instances, such as the biosensor illustrated in FIG. 7A, the spacing between materials 106 and 110 is defined as the shortest distance between (i) the portion (e.g., a point) of material 106 that is closest to material 110 and (ii) the portion (e.g., a point) of material 110 that is closest to material 106.

In certain embodiments, a plane including the top of material 106 and a plane including the top of material 110 are separated by a distance ranging from about 1 Angstrom to about 10⁵ Angstroms, from about 10 Angstroms to about 10⁵ Angstroms, from about 25 Angstroms to about 10⁴ Angstroms, or from about 40 Angstroms to about 10² Angstroms. In some embodiments of the present invention, a plane including the top of material 106 and a plane including the top of material 110 are separated by a distance that is less than 200 Angstroms, less than 150 Angstroms, less than 100 Angstroms, less than 50 Angstroms, less than 40 Angstroms, or less than 30 Angstroms. In some embodiments of the present invention, a plane including the top of material 106 and a plane including the top of material 110 are separated by a distance of between about 10 Angstroms and about 20 Angstroms, between about 20 Angstroms and about 30 Angstroms, between about 30 Angstroms and about 40 Angstroms, between about 40 Angstroms and about 50 Angstroms, between about 40 Angstroms and about 80 Angstroms, between about 70 Angstroms and about 120 Angstroms, or between about 50 Angstroms and about 150 Angstroms.

In certain embodiments, a portion (e.g., a point) of material 106 and a portion (e.g., a point) of material 110 are separated by a distance ranging from about 1 Angstrom to about 10⁵ Angstroms, from about 10 Angstroms to about 10⁵ Angstroms, from about 25 Angstroms to about 10⁴ Angstroms, or from about 40 Angstroms to about 10² Angstroms. In some embodiments of the present invention, a portion of material 106 and a portion of material 110 are separated by a distance that is less than 200 Angstroms, less than 150 Angstroms, less than 100 Angstroms, less than 50 Angstroms, less than 40 Angstroms, or less than 30 Angstroms. In some embodiments of the present invention, a portion of material 106 and a portion of material 110 are separated by a distance of between about 10 Angstroms and about 20 Angstroms, between about 20 Angstroms and about 30 Angstroms, between about 30 Angstroms and about 40 Angstroms, between about 40 Angstroms and about 50 Angstroms, between about 40 Angstroms and about 80 Angstroms, between about 70 Angstroms and about 120 Angstroms, or between about 50 Angstroms and about 150 Angstroms.

6.17 Reactive Groups and Electrolabile Masking Groups

In the present invention, reactive groups 122 and 125 are any group that is capable of binding to materials 106 and 110. In some embodiments of the present invention, reactive groups 122 and 125 are the same or different and are selected from the group consisting of a sulfate, a sulfonate, a sulfonyl moiety, a thiol, a thioether, a sulfur-containing moiety, a chalcogen-containing moiety, an amine, a carbonyl-containing moiety, a carboxylic acid, an aldehyde, a ketone, a phosphate, a phosphonate, a phosphorothioate, a pnictogen moiety, a silane, a silicon-containing moiety, an alkene, an alkyne, a hydroxyl group, and a halogen.

In some embodiments of the present invention, electrolabile masking groups 124 and 126 are the same or different and are independently selected from the group consisting of an aryl-containing moiety, a heteroaryl-containing moiety, an electron-withdrawing entity, a halogen-containing moiety, a carbonyl containing moiety, a sulfonyl-containing moiety, an arylsulfonyl group, a tribomoethyl group, and derivatives thereof.

Electrolabile masking groups of the present invention (e.g., groups 124 and 126) are any groups that can mask the reactivity of reactive groups 122 and/or 125. Representative reactive groups 122 and 125 and suitable electrolabile masking groups 124 and 126 are found in, for example, Greene and Wuts, Protective Groups in Organic Synthesis, Third Edition, 1999, John Wiley & Sons, New York, which is hereby incorporated by reference in its entirety. The following subsections provide examples of representative conditions for removal of electrolabile masking groups in accordance with the present invention. Two additional electrolabile masking groups include a S-2,4,6-trimethoxybenzyl thioether derivative, which can be removed from a thiol by electrolysis in 0.25M H₂SO₄, and a S-2-picolyl N-oxide thioether derivative, which can be removed from a thiol by electrolysis in dimethylformamide (DMF).

6.17.1 S-2,2,2-Trichloroethoxycarbonyl Derivatives

In some embodiments of the present invention, reactive group 122 is a thiol and electrolabile masking group 124 is a S-2,2,2-trichloroethoxycarbonyl derivative. In such embodiments, reactive group 122 and masking group 124 together form a thiocarbonate that can be cleaved at −1.5 volts in the presence of LiClO₄/CH₃OH to yield the reactive thiol group 122. In some embodiments of the present invention, reactive group 125 is a thiol and electrolabile masking group 126 is an S-2,2,2-trichloroethoxycarbonyl derivative. In such embodiments, reactive group 125 and masking group 126 together form a thiocarbonate that can be cleaved at −1.5 volts in the presence of LiClO₄/CH₃OH to yield the reactive thiol group 125. For information on these cleavage conditions, see Semmelhack and Heinsohn, 1972, J. Am. Chem. Soc. 94, 5139, which is hereby incorporated by reference in its entirety.

6.17.2 S-Benzyloxycarbonyl Derivatives

In some embodiments of the present invention, reactive group 122 is a thiol and electrolabile masking group 124 is a s-benzyloxycarbonyl derivative. In such embodiments, reactive group 122 and masking group 124 together form a thiocarbonate that can be cleaved at −2.6 volts in the presence of DMF and tetrabutyl ammonium chloride to yield the reactive thiol group 122. In some embodiments of the present invention, reactive group 125 is a thiol and electrolabile masking group 126 is a s-benzyloxycarbonyl derivative. In such embodiments, reactive group 125 and masking group 126 together form a thiocarbonate that can be cleaved at −2.6 volts in the presence of DMF and tetrabutyl ammonium chloride to yield the reactive thiol group 125. For information on these cleavage conditions, see Mairanovsky, 1976, Angew. Chem., Int. Ed. Engl. 15, 281, which is hereby incorporated by reference in its entirety.

6.17.3 S-Benzyl Thioether Derivatives

In some embodiments of the present invention, reactive group 122 is a thiol and electrolabile masking group 124 is an s-benzyl thioether derivative. In such embodiments, reactive group 122 and masking group 124 together form a thioether that can be cleaved at between −2.7 volts and —2.85 volts in the presence of dimethylformamide (DMF) and a suitable electrolyte such as tetraalkyl ammonium perchlorate (e.g., tetrabutyl or tetraethylammonium perchlorate). In some embodiments of the present invention, reactive group 125 is a thiol and electrolabile masking group 126 is a an s-benzyl thioether derivative. In such embodiments, reactive group 125 and masking group 126 together form a thioether that can be cleaved at between −2.7 volts and −2.85 volts in the presence of dimethylformamide (DMF) and a suitable electrolyte such as tetraalkyl ammonium perchlorate (e.g., tetrabutyl or tetraethylammonium perchlorate). For information on such cleavage conditions, see Mairanovsky, 1976, Angew. Chem., Int. Ed. Engl. 15, 281; Lelerue-Matos et al., 1991, J. Electroanal. Chem. Interfacial Electrochem. 315, 1; and U.S. Pat. No. 4,338,452 to Katner and Bogard.

6.17.4 S-Triphenylmethyl Thioether Derivatives

In some embodiments of the present invention, reactive group 122 is a thiol and electrolabile masking group 124 is an s-triphenylmethyl thioether derivative. In such embodiments, reactive group 122 and masking group 124 together form a thioether that can be cleaved at −2.6 volts in the presence of DMF and tetrabutyl ammonium chloride to yield the reactive thiol group 122. In some embodiments of the present invention, reactive group 125 is a thiol and electrolabile masking group 126 is a s-triphenylmethyl thioether derivative. In such embodiments, reactive group 125 and masking group 126 together form a thioether derivative that can be cleaved at −2.6 volts in the presence of DMF and tetrabutyl ammonium chloride to yield the reactive thiol group 125. For information on such cleavage conditions, see Mairanovsky, 1976, Angew. Chem., Int. Ed. Engl. 15, 281, which is hereby incorporated by reference in its entirety.

6.18 Intercalators

Preferred intercalating groups 128 preferentially associate with double stranded polynucleotides as compared to single stranded polynucleotides. Exemplary intercalating groups 128 comprise ethidium bromide, acridine, and derivatives of these compounds. Exemplary acridine derivatives include acridine orange, acridine yellow, 9-aminoacridine, hydrochloride hydrate, 2-aminoacridone, 9,9′-biacridyl, 9-chloroacridine, 6,9-dichloro-2-methoxyacridine, n-(1-leucyl)-2-aminoacridone, and 10-octadecyl acridine orange. Other suitable intercalators 128 include rivanol, doxorubicin, daunorubicin, actinomycin D, 7-amino Actinomycin D, ellipticine, coralyne, propidium, TAS103, berberine, distamycin, berenil, 7H-methylbenzo[e]pyrido[4,3-b]indole, meso-tetrakis(N-methyl-4pyridyl)porphine, N-methyl mesoporphyrin, diamidino-2phenylindole, 1-pyrenemethylamine hydrochloride, netropsin, hoeschst 33342, hoeschst 33258, hoeschst 8208, naphthalene diimide, and the like.

6.19 Photosensitive and Chemically Sensitive Leaving Groups

6.19.1 Representative Photosensistive Masking Groups

A wide range of photosensitive masking groups are known in the art and all such groups are encompassed within the present invention. For illustrative purposes, a few such masking groups are described herein. However, the present application is not limited to the examples provided herein.

In some embodiments of the present invention, group 302 (FIG. 3) is a photosensitive masking group. In some embodiments, the photosensitive masking group is a halogenated coumarinylmethyl halide derivative. In one embodiment of the present invention, a wide range of compounds containing at least a weakly acidic reactive group 300 (FIG. 3), such as a carboxylic acid group, a sulfonic acid group, a phosphoric acid group, a phosphonic acid group, a thiol group, a phenol group, or an amine group is caged by the reaction of a halogenated coumarinylmethyl halide derivative in the presence of the compound to be caged (Z-H), in the presence of a H-halogen scavenger, such as triethylamine, diisoproplylethylamine, 1,8-diazabicyclo[5.4.0]undec-7-ene (DBU), NaH, or Ag₂O, as depicted in the following equation:

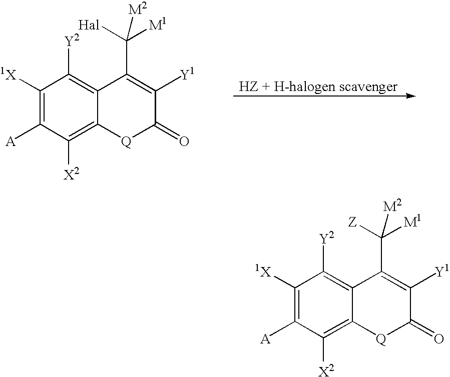

where,

A is —OH, substituted or unsubstituted alkoxy, —OC(O)CH₃, —NH₂, or —NHCH₃;

each of X¹ and X², independently, is H, Cl, Br, or I, at least one of X¹ and X² being Cl, Br, or I;

Q is —O—, —NH—, or —NCH₃—;

Y¹ is —H, —Cl, —Br, —I, —C(O)OH, —NO₂, —C(O)NHR¹, —CN, —C(O)H, —C(O)CH₃, benzoxazol-2-yl, benzothiazol-2-yl, or benzimidazol-2-yl;

Y² is —H, —C(O)OH, or —SO₃H; M¹ is —H, —CH₃, —NR2R³, —C(O)NR² R³, or —COOH;

HAL is halogen;

Z is a leaving group;

M² is —H, or Z and M² together are ═N₂, ═O, or ═NNHR¹; and

each of R¹, R², and R³, independently, is a substituted or unsubstituted moiety selected from the group consisting of a C₁₋₂₀ alkyl, a C₂₋₂₀ alkenyl, a C₂₋₂₀ alkynyl, a C₁₋₂₀ alkoxy, a C₁₋₂₀ thioalkoxy, a C₁₋₂₀ alkylsulfonyl, a C₄₋₁₆ arylsulfonyl, a C₂₋₂₀ heteroalkyl, a C₂₋₂₀ heteroalkenyl, a C₃₋₈ cycloalkyl, a C₃₋₈ cycloalkenyl, a C₄₋₁₆ aryl, a C₄₋₁₆ heteroaryl, and a C₂₋₃₀ heterocyclyl. See, for example, U.S. Pat. No. 6,472,541 to Tsien and Furuta, which is hereby incorporated by reference in its entirety.

An alkyl group is a branched or unbranched hydrocarbon that may be substituted or unsubstituted. Examples of branched alkyl groups include isopropyl, sec-butyl, isobutyl, tert-butyl, sec-pentyl, isopentyl, tert-pentyl, isohexyl. An alkenyl group contains one or more carbon-carbon double bonds. An alkynyl group contains one or more carbon-carbon triple bonds. Examples of cycloalkyl groups include cyclopropyl, cyclobutyl, cyclopentyl, cyclohexyl, cycloheptyl, and cyclooctyl. An aryl group is an aromatic ring, where the ring is made of carbon atoms. A cycloalkenyl group is a cycloalkyl containing a carbon-carbon double bond. A cyclitol radical is a cycloalkyl group having one or more hydroxyl groups (e.g., inositol).

An alkoxy group is an alkyl group linked to an oxygen atom through which it is linked to another moiety. When the leaving group is an alkoxy group, the alkoxide anion can be the conjugate base of an alcohol having a low pH. Examples of suitable alkoxy groups include —OCCl₃, and —OCF₃. An aryloxy group is an aryl group linked to an oxygen atom through which it is linked to another moiety. A thioalkoxy is an alkyl group linked to a sulfur atom through which it is linked to another moiety. An alkylsulfonyl or arylsulfonyl group is an alkyl or aryl group linked to a sulfonyl group through which it is linked to another moiety.

A heteroalkyl, a heteroalkenyl, heterocyclyl group contains at least one ring structure that contains carbon atoms and at least one heteroatom (e.g., N, O, S, or P). A heteroaryl is an aromatic heterocyclic radical. Examples of heterocyclyl radicals and heteroaryl groups include: thiazolyl, thienyl, furyl, 1-isobenzofuranyl, 2H-chromen-3-yl, 2H-pyrrolyl, N-pyrrolyl, imidazolyl, pyrazolyl, isothiazolyl, isooxazolyl, pyridyl, pyrazinyl, pyrimidinyl, pyradazinyl, indolizinyl, isoindolyl, indolyl, indazolyl, purinyl, phthalazinyl, cinnolinyl, benzoxazol-2-yl, benzothiazol-2-yl, or benzimidazol-2-yl, and pteridinyl. A heterocyclyl group may be attached to another moiety via a carbon atom or a heteroatom of the heterocyclic radical.

6.19.2 Unmasking Conditions for Photosensistive Masking Groups

A broad range of conditions can be used to remove the photosensitive masking groups of the present invention including ultraviolet light and laser light. For example, in instances where the photosensitive masking group 302 is a halogenated coumarinylmethyl halide derivative described in Section 6.19.1, the masking group can be removed using a wide range of light sources such as a 365 nm ultraviolet light from a B-100 mercury lamp (Spectronics Corp., Westbury, N.Y.) or a mode-locked titatnium-sapphire laser (Spectra-Physics TSUNAMI, pumped by a Spectra-Physics MILLENNIUM).

6.19.3 Representative Chemically Sensitive Masking Groups

A wide range of chemically sensitive masking groups are known in the art and all such groups are encompassed within the present invention. For illustrative purposes, a few such masking groups are described herein. However, the present application is not limited to the examples provided herein.

In some embodiments of the present invention, reactive group 300 (FIG. 3) is a thiol and reactive group 300 and chemically sensitive masking group 302 together form an s-alkyl thioether having the formula CₙH₂ₙ₊₁SR, where R is macromolecule 120. This thioether can be cleaved in the presence of Na/NH₃. For information on these cleavage conditions, see Lutgring et al., 1993, Bioorg. Med. Chem. Lett. 3, 739, which is hereby incorporated by reference in its entirety. Further, see Ford et al., 1994, J. Org. Chem. 59, 5955, which is hereby incorporated by reference in its entirety.

In some embodiments of the present invention, reactive group 300 (FIG. 3) is a thiol and reactive group 300 and chemically sensitive masking group 302 together form an s-benzyl thioether having the formula RSCh₂Ph, where R is macromolecule 120. This thioether can be cleaved in the presence of Na/NH₃ or Li, NH₃ and THF. For information on these cleavage conditions, see Corrie et al., 1977, J. Chem. Soc. Perkin Trans. 1, which is hereby incorporated by reference in its entirety. Further, see Koreeda and Yang, 1994, Synlett, 201, which is hereby incorporated by reference in its entirety.

In some embodiments of the present invention, reactive group 300 (FIG. 3) is a thiol and reactive group 300 and chemically sensitive masking group 302 together form an s-diphenylmethyl thioether having the formula RSCH(C₆H₅)₂, where R is macromolecule 120. This thioether can be cleaved by a number of chemical reactions, including the presence of CF₃COOH and 2.5% phenol. For information on these cleavage conditions, see Photaki et al., 1970, J. Chem. Soc. C, 2683, which is hereby incorporated by reference in its entirety.

A number of other chemically sensitive masking groups are found in Greene and Wuts, Protective Groups in Organic Synthesis, Third Edition, 1999, John Wiley & Sons, Inc., which is hereby incorporated by reference in its entirety.

6.20 Methods for Making the Biosensors of the Present Invention

The biosensors described herein can be made using the techniques disclosed in copending copending U.S. application No. 10/***,***, filed Dec. 26, 2002, titled “DEVICE STRUCTUR FOR CLOSELY SPACED ELECTRODES,” invented by Sandeep Kunwar and George T. Mathai and having attorney docket number 11210-018-999 and incorporated herein by reference in its entirety.

6.21 Analyte Detection and Quantification

This section describes a number of techniques that can be used to detect and quantify analytes in accordance with various methods of the present invention.

6.21.1 Sample Preparation

Virtually any sample containing an analyte can be analyzed using biosensors of this invention. Such samples include, but are not limited to, body fluids or tissues, water, food, blood, serum, plasma, urine, feces, tissue, saliva, oils, organic solvents, earth, water, air, or food products. In one embodiment, the sample is a biological sample. The term “biological sample”, as used herein, refers to a sample obtained from an organism or from components (e.g., cells) of an organism. In some embodiments, the sample is any biological tissue or fluid. Frequently, the sample is a “clinical sample” which is a sample derived from a patient. Such samples include, but are not limited to, sputum, cerebrospinal fluid, blood, blood fractions (e.g. serum, plasma), blood cells (e.g., white cells), tissue or fine needle biopsy samples, urine, peritoneal fluid, and pleural fluid, or cells therefrom. Biological samples may also include sections of tissues such as frozen sections taken for histological purposes.

Biological samples, (e.g. serum) may be analyzed directly or they may be subject to some preparation prior to use in the assays of this invention. Such preparation can include, but is not limited to, suspension/dilution of the sample in water or an appropriate buffer or removal of cellular debris, e.g. by centrifugation, or selection of particular fractions of the sample before analysis.

6.21.2 Sample Delivery System

The sample that includes an analyte can be introduced into the biosensors of this invention according to standard methods well known to those of skill in the art. Thus, for example, the sample can be introduced into the channel through an injection port, such as those used in high pressure liquid chromatography systems.

6.21.3 Sample Reaction with a Macromolecule

The analyte containing sample is provided to one or more devices 144 of a biosensor of the present invention in conditions that facilitate binding of the analyte to one or more macromolecules 120 that are bound to materials 106 and 110 of the devices 144. Thus, for example, when macromolecules 120 bound to materials 106 and 110 in devices 144 of a biosensor are antibodies or proteins, reaction conditions are provided at that facilitate antibody binding. Such reaction conditions are well known to those of skill in the art. See, for example, Coligan, 1991, Current Protocols in Immunology, Wiley/Greene, NY; Harlow and Lane, 1989, Antibodies: A Laboratory Manual Cold Spring Harbor Press, NY; Stites et al. (eds.) Basic and Clinical Immunology (4^{th} ed.), Lange Medical Publications, Los Altos, Calif., and references cited therein; Goding, 1986, Monoclonal Antibodies: Principles and Practice (2^{nd} ed.) Academic Press, New York, N.Y.; and Kohler and Milstein, 1975, Nature 256: 495-497.

Similarly, where macromolecule 120 is a nucleic acid, the biosensor is maintained under conditions that facilitate binding of the target nucleic acid (analyte) to the macromolecule 120 that is bound to materials 106 and 110 in target devices 144 of the biosensor. Stringency of the reaction can be adjusted until the sensor shows adequate/desired specificity and selectivity. Conditions suitable for nucleic acid hybridization are well known to those of skill in the art. See, for example, Berger and Kimmel, Guide to Molecular Cloning Techniques, Methods in Enzymology, 152 Academic Press, Inc., San Diego, Calif.; Sambrook et al., 1989, Molecular Cloning—A Laboratory Manual (2^{nd} ed.) Vol. 1-3, Cold Spring Harbor Laboratory, Cold Spring Harbor Press, N.Y.; Ausubel et al., 1994, Current Protocols in Molecular Biology, Greene Publishing Associates, Inc. and John Wiley & Sons, Inc., New York; U.S. Pat. No. 5,017,478; and European Patent Number 0,246,864. Once the analyte is bound to the macromolecule 120 in one or more devices 144 of the biosensor, the sensor is optionally dehydrated and then read.

6.21.4 Analyte Detection and Quantification

Once introduced into the devices 144 of the present invention, the sample is detected/quantified using standard methods such as amperometry, voltammetry, coulometry. In some embodiments, the measurement results are compared to a standard curve, e.g. a series or measurement results are plotted as a function of analyte concentration. This permits determination of analyte concentration.

6.22 Cassettes

In certain embodiments, this invention provides a cassette. In some embodiments, a cassette comprises one or more devices 144 or arrays of devices 144. In some embodiments, a cassette comprises a plurality of macromolecules 120, where each macromolecule 120 is attached to a material 106/material 110 pair in a device 144. In such embodiments, material 106 and material 110 serve are electrodes. In some embodiments, counter electrodes are provided.

In one embodiment of the present invention, a cassette or apparatus of the present invention comprises a sample port and/or reservoir and one or more channels for sample delivery into the devices 144 present in the cassette. The means for sample delivery can be stationary or movable and can be any known in the art, including, but not limited to, one or more inlets, holes, pores, channels, pipes, microfluidic guides (e.g., capillaries), tubes. The one or more channels in the cassette can take the form of a channel network. This channel network might include microchannels. Reservoirs in which the desired analysis takes place are typically included within a given channel network. Additionally, the channel network optionally includes channels for delivering reagents, buffers, diluents, sample material and the like to the analysis channels.

The cassettes of the present invention optionally include separation channels or matrices separating/fractionating materials transported down the length of these channels, for analysis. For example, such separation channels or matrices may separate particles within a fluid by size or charge. Suitable separation matrices for use in such channels or matrices include, for example, GeneScan™ polymers (Perkin Elmer-Applied Biosystems Division, Foster City, Calif.). In other embodiments, analysis channels are devoid of any separation matrix, and instead, merely provide a channel within which an interaction, reaction etc., takes place. Examples of microfluidic devices incorporating such analysis channels are described in, for example, PCT Application No. WO 98/00231, and U.S. Pat. No. 5,976,336.

Fluids can be moved through the cassette channel system by a variety of well known methods. Some examples include pumps, pipettes, syringes, gravity flow, capillary action, wicking, electrophoresis, electroosmosis, pressure, and vacuum. The means used for fluid movement may be located on the cassette or on a separate unit.

The test sample can be placed on all of the devices 144 in a cassette. Alternatively, a sample may be placed on particular devices 144 in a cassette. One method for placing a sample on select devices 144 in a cassette is the use of capillary fluid transport means. Alternatively, samples may be placed on the devices 144 by an automatic pipetter for delivery of fluid samples directly to sensor array, or into a reservoir in a cassette or cassette holder for later delivery directly to devices 144 in a cassette.

The cassettes of the present invention can be fabricated from a wide variety of materials including, but not limited to glass, plastic, ceramic, polymeric materials, elastomeric materials, metals, carbon or carbon containing materials, alloys, composite foils, silicon and/or layered materials. Supports may have a wide variety of structural, chemical and/or optical properties. They may be rigid or flexible, flat or deformed, transparent, translucent, partially or fully reflective or opaque and may have composite properties, regions with different properties, and may be a composite of more than one material.

Reagents for conducting assays may be stored on the cassette and/or in a separate container. Reagents can be stored in a dry and/or wet state. In one embodiment, dry reagents in the cassette are rehydrated by the addition of a test sample. In a different embodiment, the reagents are stored in solution in “blister packs” that are burst open due to pressure from a movable roller or piston. The cassettes may contain a waste compartment or sponge for the storage of liquid waste after completion of the assay. In one embodiment, the cassette includes a device for preparation of the biological sample to be tested. Thus, for example, a filter may be included for removing cells from blood. In another example, the cassette may include a device such as a precision capillary for the metering of sample.

A cassette or apparatus of the present invention can, optionally, comprise reference electrodes, e.g., Ag/AgCl or a saturated calomel electrode (SCE) and/or various biasing/counter-electrodes. The cassette can also comprise more one layer of electrodes. Thus, for example, different electrode sets (e.g. arrays of sensor elements) can exist in different lamina of the cassette and thus form a three dimensional array of sensor elements.

6.23 Integrated Assay Device/Apparatus

State-of-the-art chemical analysis systems for use in chemical production, environmental analysis, medical diagnostics and basic laboratory analysis are often capable of complete automation. Such total analysis systems (TAS) automatically perform functions ranging from introduction of sample into the system, transport of the sample through the system, sample preparation, separation, purification and detection, including data acquisition and evaluation. See, for example, Fillipini et al., 1991, J. Biotechnol. 18: 153; Garn et al, 1989, Biotechnol. Bioeng. 34: 423; Tshulena, 1988, Phys. Scr. T23: 293; Edmonds, 1985, Trends Anal. Chem. 4: 220; Stinshoff et al., 1985, Anal. Chem. 57:114R; Guibault, 1983, Anal. Chem Symp. Ser. 17: 637; Widmer, 1983, Trends Anal. Chem. 2: 8.

Recently, sample preparation technologies have been successfully reduced to miniaturized formats. Thus, for example, gas chromatography (Widmer et al., 1984, Int. J. Environ. Anal. Chem. 18: 1), high pressure liquid chromatography (Muller et al., 1991, J. High Resolut. Chromatogr. 14: 174) and capillary electrophoresis (Manz et al., 1992, J. Chromatogr. 593: 253) have been reduced to miniaturized formats. Similarly, in certain embodiments, the present invention provides an integrated assay device (e.g., a TAS) for detecting and/or quantifying one or more analytes using the devices 144, device 144 arrays, or cassettes described above.

Thus, in certain embodiments, the cassettes of this invention are designed so that they insert into an apparatus that contains means for reading one or more devices 144 in the cassette. The apparatus optionally includes means for applying one or more test samples onto the devices 144 of the cassette or into a receiving port or reservoir associated with the cassette. Such an apparatus may be derived from conventional apparatus suitably modified according to the invention to conduct a plurality of assays based on a support or cassette. Such modifications may include the provision for sample and/or cassette handling, multiple sample delivery, multiple electrode reading by a suitable detector, and signal acquisition and processing means.

Some apparatus in accordance with the present invention includes instrumentation suitable for performing electrochemical measurements and associated data acquisition and subsequent data analysis. One such apparatus also provide means to hold cassettes, optionally provide reagents and/or buffers and to provide conditions compatible with binding agent/target analyte binding reactions. In addition to such features, one such apparatus also includes an electrode contact means that is able to electrically connect the array of separately addressable electrode connections of the cassette to an electronic-voltage/waveform generator, e.g., a potentiostat. The waveform generator delivers signals sequentially or simultaneously to independently read a plurality of sensor elements in the cassette. In some embodiments, the apparatus optionally comprises a digital computer or microprocessor to control the functions of the various components of the apparatus. In some embodiments, the apparatus also comprises signal-processing means. In one exemplary embodiment, the signal processing means comprises a digital computer for transferring, recording, analyzing and/or displaying the results of each assay.

The sensor element arrays of this invention are particularly well suited for use as detectors in “low sample volume” instrumentation. Such applications include, but are not limited to, genomic applications, such as monitoring gene expression in plants or animals, parallel gene expression studies, high throughput screening, clinical diagnosis, single nucleotide polymorphism (SNP) screening, and genotyping. Some embodiments include miniaturized molecular assay systems (e.g., “labs-on-a-chip”), that are capable of performing thousands of analyses simultaneously.

6.24 Kits

In some embodiments, this invention provides kits for practice of the methods and/or assembly of the inventive devices. Preferred kits comprise a container containing one or more devices 144. In certain embodiments, the kits optionally include one or more reagents and/or buffers for use with the inventive biosensors. In some embodiments, the kits include materials for sample acquisition and data processing.

In some embodiments, the kits include instructional materials containing directions (e.g., protocols) for the practice of the assay methods of this invention and the use of the cassettes described herein, methods of assembling sensor elements into various instruments. While the instructional materials typically comprise written or printed materials, they are not so limited. Any medium capable of storing such instructions and communicating them to a user is contemplated by this invention. Such media includes, but is not limited to, electronic storage media (e.g., magnetic discs, tapes, cartridges, chips) and optical media (e.g., CD ROM). Such media may include addresses to Internet sites that provide such instructional materials.

6.25 Monitoring Electron Transfer Through Bound Macromolecule/Analyte Complexes

In a some embodiments of the present invention, electron transfer through bound macromolecule 120/target analyte complexes is performed using amperometric detection. In some embodiments, the amperometric detector used for such detection resembles the numerous enzyme-based biosensors currently used to monitor blood glucose, for example. This method of detection involves applying a potential (as compared to a separate reference electrode) between materials 106 and 110 in a given device 144 in an inventive biosensor. Electron transfer of differing efficiencies is induced in samples in the presence or absence of analyte. For example, in the case where macromolecule 120 is a single stranded nucleic acid and the analyte is the complement to the single stranded nucleic acid, bound macromolecule 120 exhibits a different current than the corresponding bound macromolecule/analyte complex. The differing efficiencies of electron transfer result in differing currents being generated.

In some embodiments, the amperometric devices used herein use sensitive (nanoamp to picoamp) current detection and include a means of controlling the voltage potential, such as a potentiostat. In other embodiments, alternative electron detection methods are utilized. For example, potentiometric (or voltammetric) measurements involving non-faradaic (no net current flow) processes that are traditionally utilized in pH detectors can be used to monitor electron transfer through bound macromolecule 120/target analyte complexes. In addition, other properties of insulators, such as resistance, and of conductors, such as conductivity, impedance and capacitance, can be used to monitor electron transfer through bound macromolecule 120/target analyte complexes. Finally, any system that generates a current, such as electron transfer, also generates a magnetic field. Therefore, magnetic fields can be monitored in some embodiments of the present invention.

In some embodiments, the relatively fast rates of electron transfer through the binding agent/target analyte complex facilitates analysis of the frequency (time) domain and thereby dramatically improves signal to noise (S/N) ratios. Thus, in certain embodiments, electron transfer is initiated and detected using alternating current (AC) methods. In general, the use of AC techniques can result in good signals and low background noise. Without being bound by any particular theory, it is believed that there are a number of possible contributors to background noise, or “parasitic” signals, i.e. detectable signals that are inherent to the system but are not the result of the presence of the target sequence. However, all of the contributors to parasitic noise generally give relatively fast signals. That is, the rate of electron transfer through the bound macromolecule 120/target analyte complex is generally significantly slower than the rate of electron transfer of the parasitic components, such as the contribution of charge carriers in solution, and other “short circuiting” mechanisms. As a result, the parasitic components are generally all phase related. That is, they exhibit a constant phase delay or phase shift that will scale directly with frequency. The bound macromolecule 120/target analyte complex, in contrast, exhibits a time delay between the input and output signals that is independent of frequency. Thus, signal produced by analyte binding will remain relatively constant and relatively large as compared to parasitic background. As a consequence, at different frequencies, the phase of the system will change. This is very similar to the time domain detection used in fluorescent systems. This difference can be exploited in various methods of the present invention to decrease the signal to noise ratio. Accordingly, the preferred detection methods comprise applying an AC input signal to a bound macromolecule 120/target analyte complex. The presence of the bound macromolecule 120/target analyte complex is detected via an output signal characteristic of electron transfer through the bound macromolecule 120/target analyte complex. That is, the output signal is characteristic of the bound macromolecule 120/target analyte complex rather than the parasitic components or unbound binding agent. Thus, for example, the output signal will exhibit a time delay dependent on the rate of electron transfer through the bound macromolecule 120/target analyte complex.

In some embodiments of the present invention, the input signals are applied at a plurality of frequencies, since this again allows the distinction between true signal and noise. “Plurality” in this context means at least two, and preferably more, frequencies. In general, the AC frequencies will range from about 0.1 Hz to about 10 mHz or from about 1 Hz to 100 KHz. In certain preferred embodiments, data analysis is preformed in the time domain (frequency domain). Thus, for example, cyclic voltammetry is performed where the signal is analyzed at a harmonic of the fundamental frequency. Such measurements can significantly improve the signal to noise (S/N) ratio.

In some embodiments of the present invention, a cyclic (e.g., sinusoidal sweeping voltage) is applied to materials 106 and 110. The response of the bound macromolecule 120/target analyte complex to the sinusoidal voltage is selectively detected at a harmonic of the fundamental frequency of the cyclic voltage rather than at the fundamental frequency. As a result, a complete frequency spectrum can be obtained within one cycle.

6.26 Measuring Analyte Binding Events

The biosensors of the present invention may be used to detect macromolecule 120/analyte binding events. Such binding events may arise through, for example, ligand/receptor, enzyme/substrate, DNA/DNA, DNA/RNA, RNA/RNA, nucleic acid/protein interactions.

In one embodiment of the present invention, macromolecule 120 is a single stranded DNA bound two an electrode pair (materials 106 and 110) in a given device 144 in the biosensor and the analyte is a single stranded DNA. In this embodiment, an alternating current (AC) conductance test is used to determine whether a binding event has occurred in the device 144. This is done by measuring the AC conductance G_{AC}=ε″A/d at the device 144, where A is the effective area of one electrode and d is the effective distance between electrodes. At the relaxation frequency of a given double stranded DNA molecule (e.g., macromolecule 120 bound to an analyte to form a double stranded nucleic acid) should be different (e.g., larger) than the conductance when no analyte bound to the macromolecule 120 bound in the device 144. A pulsed or frequency-scanned waveform is applied across the electrode pair in the device 144. The presence of hybridized DNA is detected at a resonant frequency of DNA. An LCR meter may be used to measure G or R=1/G at a discrete frequency. Alternatively, G can be measured as a function of frequency.

In another embodiment, a frequency scanned or chirped voltage waveform Vᵢ is applied across the electrodes at each site and the resultant response waveform Vₒ, depending upon whether frequency is increasing or decreasing, is analyzed to determine the presence of hybridized DNA as indicated by a maxima at a hybridized DNA frequency. The measurement of the relaxation frequency of the hybridized DNA using a frequency-scanned waveform gives additional information about the properties of the hybridized DNA, e.g., crosslinked versus non-crosslinked.

6.27 High Stringency

High stringency conditions are known in the art; see for example Maniatis et al., Molecular Cloning: A Laboratory Manual, 2d Edition, 1989, and Short Protocols in Molecular Biology, ed. Ausubel et al., both of which are hereby incorporated by reference in their entireties. High stringency conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point™ for the specific sequence at a defined ionic strength pH. The Tₘ is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30 C. for short probes (e.g. 10 to 50 nucleotides) and at least about 60° C. for long probes (e.g. greater than 50 nucleotides). Stringent conditions may also be achieved with the addition of destabilizing agents, such as formamide.

By way of example and not limitation, procedures using conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency that may be used depend on the nature of the nucleic acid (e.g. length, GC content, etc.) and the purpose of the hybridization (detection, amplification, etc.) and are well known in the art. For example, stringent hybridization of an oligonucleotide of approximately 15 to 40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C. The skilled artisan will recognize that the temperature, salt concentration, and chaotrope composition of hybridization and wash solutions may be adjusted as necessary according to factors such as the length and nucleotide base composition of the probe. Another embodiment of the present invention provides a nucleic acid that hybridizes under conditions of moderate stringency to about nucleotide 760 through about nucleotide 1215 of SEQ ID NO: 2. Still another embodiment of the present invention provides a nucleic acid that hybridizes under conditions of moderate stringency to a polynucleotide that is complementary to nucleotides 760 through 1215 of SEQ ID NO: 2. As used herein, conditions of moderate stringency, as known to those having ordinary skill in the art, and as defined by Sambrook et al., Molecular Cloning: A Laboratory Manual, 2^{nd} Ed. Vol. 1, pp. 1.101-104, Cold Spring Harbor Laboratory Press, 1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, or Stark's solution, in 50% formamide at 42° C.), and washing conditions of about 60° C., 0.5×SSC, 0.1% SDS. See also, Ausubel et al., eds., in the Current Protocols in Molecular Biology series of laboratory technique manuals, ©1987-1997, Current Protocols, ©1994-1997, John Wiley and Sons, Inc.). The skilled artisan will recognize that the temperature, salt concentration, and chaotrope composition of hybridization and wash solutions may be adjusted as necessary according to factors such as the length and nucleotide base composition of the probe.

6.28 Intermediate Stringency

As used herein, conditions of moderate stringency, as known to those having ordinary skill in the art, and as defined by Sambrook et al., Molecular Cloning: A Laboratory Manual, 2^{nd} Ed. Vol. 1, pp. 1.101-104, Cold Spring Harbor Laboratory Press, 1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, or Stark's solution, in 50% formamide at 42° C.), and washing conditions of about 60° C., 0.5×SSC, 0.1% SDS. See also, Ausubel et al., eds., in the Current Protocols in Molecular Biology series of laboratory technique manuals, ©1987-1997, Current Protocols, ©1994-1997, John Wiley and Sons, Inc.). The skilled artisan will recognize that the temperature, salt concentration, and chaotrope composition of hybridization and wash solutions may be adjusted as necessary according to factors such as the length and nucleotide base composition of the probe.

6.29 Low Stringency

By way of example and not limitation, procedures using conditions of low stringency for regions of hybridization of over 90 nucleotides are as follows (see also Shilo and Weinberg, 1981, Proc. Natl. Acad. Sci. U.S.A. 78, 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency that may be used are well known in the art (e.g., as employed for cross-species hybridizations).

### 7.0 REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those of skill in the art. The specific embodiments described herein are offered by way of example only, and the invention is to be limited only by the terms of the appended claims, along with the full scope of equivalents to which such claims are entitled.

What is claimed:

1. A method of electrically coupling an electrode pair in a plurality of electrode pairs in a biosensor with a macromolecule, wherein
(i) said electrode pair comprises a first electrode and a second electrode;
(ii) a first portion of said macromolecule is derivatized with a first reactive group and a second portion of said macromolecule is derivatized with a second reactive group; and
(iii) said first reactive group is masked with a first electrolabile masking group and said second reactive group is masked with a second electrolabile masking group,
the method comprising:
(a) applying a first voltage at said first electrode in said electrode pair under conditions that are sufficient to unmask said first reactive group, wherein said unmasked first reactive group binds to said first electrode thereby linking said macromolecule to said first electrode; and
(b) applying a second voltage at said second electrode in said electrode pair under conditions that are sufficient to unmask said second reactive group, wherein said unmasked second reactive group binds to said second electrode thereby electrically coupling said electrode pair in said biosensor with said macromolecule.

2. The method of claim 1 wherein said first voltage and said second voltage are different.

3. The method of claim 1 wherein said first electrode and said second electrode are separated by a distance that is between 10 Angstroms and 10,000 Angstroms.

4. The method of claim 1 wherein said first electrode and said second electrode are separated by a distance that is between 30 Angstroms and 500 Angstroms.

5. The method of claim 1 wherein said first electrode and said second electrode are separated by a distance that is between 50 Angstroms and 200 Angstroms.

6. The method of claim 1 wherein said first electrode and said second electrode have a resistivity of less than 10⁻⁴ ohm meters.

7. The method of claim 1 wherein said first electrode and said second electrode are each made from the same or different material that are each independently selected from the group consisting of silicon, dense silicon carbide, boron carbide, Fe₃O₄, germanium, silicone germanium, silicon carbide, polysilicon, tungsten carbide, titanium carbide, indium phosphide, gallium nitride, gallium phosphide, aluminum phosphide, aluminum arsenide, mercury cadmium telluride, tellurium, selenium, ZnS, ZnO, ZnSe, CdS, ZnTe, GaSe, CdSe, CdTe, GaAs, InP, GaSb, InAs, Te, PbS, InSb, InSb, PbTe, PbSe, tungsten disulfide.

8. The method of claim 1 wherein said first electrode and said second electrode are made of a metal.

9. The method of claim 1 wherein said first electrode and said second electrode are each made from the same or different material that are each independently selected from the group consisting of ruthenium, cobalt, rhodium, rubidium, lithium, sodium potassium, vanadium, cesium, chromium, molybdenum, silicon, germanium, aluminum, iridium, nickel, palladium, platinum, iron, copper, titanium, tungsten, silver, gold, zinc, cadmium, indium tin oxide, carbon, and carbon nanotube and an alloy thereof.

10. The method of claim 1 wherein said macromolecule comprises a nucleic acid, a protein, a polypeptide, a peptide, an antibody, a carbohydrate, a polysaccharide, a lipid, a fatty acid or a sugar.

11. The method of claim 1 wherein said first reactive group and said second reactive group are the same or different material and are each independently selected from the group consisting of a sulfate, a sulfonate, a sulfonyl moiety, a thiol, a thioether, a sulfur-containing moiety, a chalcogen-containing moiety, an amine, a carbonyl-containing moiety, a carboxylic acid, an aldehyde, a ketone, a phosphate, a phosphonate, a phosphorothioate, a pnictogen moiety, a silane, a silicon-containing moiety, an alkene, an alkyne, a hydroxyl group, and a halogen.

12. The method of claim 1 wherein said first reactive group and said second reactive group are each a thiol.

13. The method of claim 12 wherein said first electrolabile masking group and said second electrolabile masking group are different and are each independently selected from the group consisting of an S-2,2,2-trichloroethoxycarbonyl derivative, an S-benzyloxycarbonyl derivative, an S-benzyl thioether derivative, an S-triphenylmethyl thioether derivative, an S-2,4,6-trimethoxybenzyl thioether derivative, and an S-2-picolyl N-oxide thioether derivative.

14. The method of claim 1, further comprising
(c) testing for background conductance between said first electrode and said second electrode;
(d) exposing said electrode pair to a solution that potentially comprises an analyte for a period of time;
(e) drying said electrode pair; and
(f) measuring a current through said electrode pair.

15. The method of claim 14 wherein said period of time is less than 30 minutes.

16. The method of claim 14 wherein said period of time is between one minute and one hour.

17. The method of claim 14 wherein said period of time is between one hour and fifteen hours.

18. The method of claim 14 wherein said macromolecule is a single stranded nucleic acid and said analyte is a single stranded nucleic acid that has sufficient complementarity to said macromolecule to bind to said macromolecule in said exposing step.

19. The method of claim 14 wherein said macromolecule is a single stranded nucleic acid and said analyte is a single stranded nucleic acid that has sufficient complementarity to said macromolecule to bind to said macromolecule under conditions of high stringency.

20. The method of claim 14 wherein said macromolecule is a single stranded nucleic acid and said analyte is a single stranded nucleic acid that has sufficient complementarity to said macromolecule to bind to said macromolecule under conditions of intermediate stringency.

21. The method of claim 14 wherein said macromolecule is a single stranded nucleic acid and said analyte is a single stranded nucleic acid that has sufficient complementarity to said macromolecule to bind to said macromolecule under conditions of low stringency.

22. The method of claim 14 wherein said drying step (e) comprises blowing nitrogen or argon gas on said electrode pair.

23. The method of claim 14 wherein said measuring step (f) comprises quantifying a current across said electrode pair when a voltage of between ±5 volts is applied across said electrode pair.

24. The method of claim 14 wherein said analyte comprises a whole cell, a subcellular particle, a virus, a prion, a viroid, a nucleic acid, a protein, an antigen, a lipoprotein, a lipopolysaccharide, a lipid, a glycoproteins, a carbohydrate moiety, a cellulose derivative, an antibody, a fragment of an antibody, a peptide, a hormone, a pharmacological agent, a cellular component, an organic compound, a non-biological polymer, a synthetic organic molecule, an organo-metallic compound, or an inorganic molecule.

25. The method of claim 1 wherein said applying step (a) and said applying step (b) are repeated with a different electrode pair in said plurality of electrode pairs using a different macromolecule, wherein
(i) said different electrode pair comprises a first electrode and a second electrode;
(ii) a first portion of said different macromolecule is derivatized with a third reactive group and a second portion of said macromolecule is derivatized with a fourth reactive group; and
(iii) said third reactive group is masked with a third electrolabile masking group and said fourth reactive group is masked with a fourth electrolabile masking group.

26. The method of claim 1 wherein said applying step (a) is repeated with a different electrode pair in said plurality of electrode pairs using a different macromolecule prior to said applying step (b), wherein
(i) said different electrode pair comprises a first electrode and a second electrode;
(ii) a first portion of said different macromolecule is derivatized with a third reactive group and a second portion of said macromolecule is derivatized with a fourth reactive group; and
(iii) said third reactive group is masked with a third electrolabile masking group and said fourth reactive group is masked with a fourth electrolabile masking group.

27. A method of electrically coupling an electrode pair in a plurality of electrode pairs in a biosensor with a macromolecule, wherein
(i) said electrode pair comprises a first electrode and a second electrode;
(ii) an intercalator is covalently linked to said second electrode; and
(iii) a portion of said macromolecule is derivatized with a reactive group that is masked with an electrolabile masking group,
the method comprising:
(a) applying a voltage at said first electrode in said electrode pair under conditions that are sufficient to unmask said reactive group, wherein said unmasked reactive group binds to said first electrode thereby linking said macromolecule to said first electrode; and
(b) exposing said electrode pair to a solution that potentially comprises an analyte for a period of time, wherein, when said analyte binds to said macromolecule to form a complex comprising said macromolecule and said analyte, said intercalator binds to said complex thereby electrically connecting said electrode pair.

28. The method of claim 27 wherein said first electrode and said second electrode are separated by a distance that is between 10 Angstroms and 10,000 Angstroms.

29. The method of claim 27 wherein said first electrode and said second electrode are separated by a distance that is between 30 Angstroms and 500 Angstroms.

30. The method of claim 27 wherein said first electrode and said second electrode are separated by a distance that is between 50 Angstroms and 200 Angstroms.

31. The method of claim 27 wherein said first electrode and said second electrode have a resistivity of less than 10⁻⁴ ohm meters.

32. The method of claim 27 wherein said first electrode and said second electrode are each made from the same or different material that are each independently selected from the group consisting of silicon, dense silicon carbide, boron carbide, Fe₃O₄, germanium, silicone germanium, silicon carbide, polysilicon, tungsten carbide, titanium carbide, indium phosphide, gallium nitride, gallium phosphide, aluminum phosphide, aluminum arsenide, mercury cadmium telluride, tellurium, selenium, ZnS, ZnO, ZnSe, CdS, ZnTe, GaSe, CdSe, CdTe, GaAs, InP, GaSb, InAs, Te, PbS, InSb, InSb, PbTe, PbSe, tungsten disulfide.

33. The method of claim 27 wherein said first electrode and said second electrode are made of a metal.

34. The method of claim 27 wherein said first electrode and said second electrode are each made from the same or different material that are each independently selected from the group consisting of ruthenium, cobalt, rhodium, rubidium, lithium, sodium potassium, vanadium, cesium, chromium, molybdenum, silicon, germanium, aluminum, iridium, nickel, palladium, platinum, iron, copper, titanium, tungsten, silver, gold, zinc, cadmium, indium tin oxide, carbon, and carbon nanotube and an alloy thereof.

35. The method of claim 27 wherein said macromolecule comprises a nucleic acid, a protein, a polypeptide, a peptide, an antibody, a carbohydrate, a polysaccharide, a lipid, a fatty acid or a sugar.

36. The method of claim 27 wherein said reactive group is selected from the group consisting of a sulfate, a sulfonate, a sulfonyl moiety, a thiol, a thioether, a sulfur-containing moiety, a chalcogen-containing moiety, an amine, a carbonyl-containing moiety, a carboxylic acid, an aldehyde, a ketone, a phosphate, a phosphonate, a phosphorothioate, a pnictogen moiety, a silane, a silicon-containing moiety, an alkene, an alkyne, a hydroxyl group, and a halogen.

37. The method of claim 27 wherein said reactive group is a thiol.

38. The method of claim 27 wherein said electrolabile masking group is selected from the group consisting of an S-2,2,2-trichloroethoxycarbonyl derivative, an S-benzyloxycarbonyl derivative, an S-benzyl thioether derivative, an S-triphenylmethyl thioether derivative, an S-2,4,6-trimethoxybenzyl thioether derivative, and an S-2-picolyl N-oxide thioether derivative.

39. The method of claim 27 wherein said period of time is less 30 minutes.

40. The method of claim 27 wherein said period of time is between one minute and one hour.

41. The method of claim 27 wherein said period of time is between one hour and fifteen hours.

42. The method of claim 27 wherein said macromolecule is a single stranded nucleic acid and said analyte is a single stranded nucleic acid that has sufficient complementarity to said macromolecule to bind to said macromolecule in said exposing step.

43. The method of claim 27 wherein said macromolecule is a single stranded nucleic acid and said analyte is a single stranded nucleic acid that has sufficient complementarity to said macromolecule to bind to said macromolecule under conditions of high stringency.

44. The method of claim 27 wherein said macromolecule is a single stranded nucleic acid and said analyte is a single stranded nucleic acid that has sufficient complementarity to said macromolecule to bind to said macromolecule under conditions of intermediate stringency.

45. The method of claim 27 wherein said macromolecule is a single stranded nucleic acid and said analyte is a single stranded nucleic acid that has sufficient complementarity to said macromolecule to bind to said macromolecule under conditions of low stringency.

46. The method of claim 27 wherein said analyte comprises a whole cell, a subcellular particle, a virus, a prion, a viroid, a nucleic acid, a protein, an antigen, a lipoprotein, a lipopolysaccharide, a lipid, a glycoproteins, a carbohydrate moiety, a cellulose derivative, an antibody, a fragment of an antibody, a peptide, a hormone, a pharmacological agent, a cellular component, an organic compound, a non-biological polymer, a synthetic organic molecule, an organo-metallic compound, or an inorganic molecule.

47. The method of claim 27 wherein said intercalator comprises ethidium, an ethidium derivative, an ethidium complex, acridine, an acridine derivative or an acridine complex.

48. The method of claim 27 wherein said intercalator comprises acridine orange, acridine yellow, 9-aminoacridine, hydrochloride hydrate, 2-aminoacridone, 9,9′-biacridyl, 9-chloroacridine, 6,9-dichloro-2-methoxyacridine, n-(l-leucyl)-2-aminoacridone, 10-octadecyl acridine orange, rivanol, doxorubicin, daunorubicin, actinomycin D, 7-amino Actinomycin D, ellipticine, coralyne, propidium, TAS 103, berberine, distamycin, berenil, 7H-methylbenzo[e]pyrido[4,3-b]indole, meso-tetrakis(N-methyl-4pyridyl)porphine, N-methyl mesoporphyrin, diamidino-2phenylindole, 1-pyrenemethylamine hydrochloride, netropsin, hoeschst 33342, hoeschst 33258, hoeschst 8208, naphthalene diimide, or ethidium bromide.

49. The method of claim 27 further comprising:
(c) drying said electrode pair; and
(d) measuring a current through said electrode pair.

50. The method of claim 49 wherein said drying step (c) comprises blowing nitrogen or argon gas on said electrode pair.

51. The method of claim 49 wherein said measuring step (d) comprises quantifying a current across said electrode pair when a voltage of between ±5 volts is applied across said electrode pair.

52. The method of claim 27 wherein said applying step (a) is repeated, before said exposing step (b), with a different electrode pair in said plurality of electrode pairs using a different macromolecule, wherein
(i) said different electrode pair comprises a first electrode and a second electrode;
(ii) a portion of said different macromolecule is derivatized with a reactive group that is masked with a electrolabile masking group.

53. The method of claim 27 wherein said applying step (a) and said exposing step (b) are repeated with a different electrode pair in said plurality of electrode pairs using a different macromolecule, wherein
(i) said different electrode pair comprises a first electrode and a second electrode; and
(ii) a portion of said different macromolecule is derivatized with a reactive group that is masked with a electrolabile masking group.

54. A method of electrically coupling an electrode pair in a plurality of electrode pairs in a biosensor with a macromolecule, wherein
(i) said electrode pair comprises a first electrode and a second electrode;
(ii) a first portion of said macromolecule is derivatized with a first reactive group and a second portion of said macromolecule is derivatized with a second reactive group; and
(iii) said first reactive group is masked with an electrolabile masking group and said second reactive group is masked with a photosensitive or chemically sensitive masking group,
the method comprising:
(a) applying a voltage at said first electrode in said electrode pair under conditions that are sufficient to unmask said first reactive group, wherein said unmasked first reactive group binds to said first electrode thereby linking said macromolecule to said first electrode; and
(b) exposing said electrode pair to a light source or a chemical thereby unmasking said second reactive group, wherein said unmasked second reactive group binds to said second electrode thereby electrically coupling said electrode pair in said biosensor with said macromolecule.

55. The method of claim 54 wherein said first electrode and said second electrode are separated by a distance that is between 10 Angstroms and 10,000 Angstroms.

56. The method of claim 54 wherein said first electrode and said second electrode are separated by a distance that is between 30 Angstroms and 500 Angstroms.

57. The method of claim 54 wherein said first electrode and said second electrode are separated by a distance that is between 50 Angstroms and 200 Angstroms.

58. The method of claim 54 wherein said first electrode and said second electrode have a resistivity of less than 10⁻⁴ ohm meters.

59. The method of claim 54 wherein said first electrode and said second electrode are each made from the same or different material that are each independently selected from the group consisting of silicon, dense silicon carbide, boron carbide, Fe₃O₄, germanium, silicone germanium, silicon carbide, polysilicon, tungsten carbide, titanium carbide, indium phosphide, gallium nitride, gallium phosphide, aluminum phosphide, aluminum arsenide, mercury cadmium telluride, tellurium, selenium, ZnS, ZnO, ZnSe, CdS, ZnTe, GaSe, CdSe, CdTe, GaAs, InP, GaSb, InAs, Te, PbS, InSb, InSb, PbTe, PbSe, tungsten disulfide.

60. The method of claim 54 wherein said first electrode and said second electrode are made of a metal.

61. The method of claim 54 wherein said first electrode and said second electrode are each made from the same or different material that are each independently selected from the group consisting of ruthenium, cobalt, rhodium, rubidium, lithium, sodium potassium, vanadium, cesium, chromium, molybdenum, silicon, germanium, aluminum, iridium, nickel, palladium, platinum, iron, copper, titanium, tungsten, silver, gold, zinc, cadmium, indium tin oxide, carbon, and carbon nanotube and an alloy thereof.

62. The method of claim 54 wherein said macromolecule comprises a nucleic acid, a protein, a polypeptide, a peptide, an antibody, a carbohydrate, a polysaccharide, a lipid, a fatty acid or a sugar.

63. The method of claim 54 wherein said first reactive group and said second reactive group are the same or different material and are each independently selected from the group consisting of a sulfate, a sulfonate, a sulfonyl moiety, a thiol, a thioether, a sulfur-containing moiety, a chalcogen-containing moiety, an amine, a carbonyl-containing moiety, a carboxylic acid, an aldehyde, a ketone, a phosphate, a phosphonate, a phosphorothioate, a pnictogen moiety, a silane, a silicon-containing moiety, an alkene, an alkyne, a hydroxyl group, and a halogen.

64. The method of claim 54 wherein said first reactive group and said second reactive group are each a thiol.

65. The method of claim 54 wherein said electrolabile masking group is selected from the group consisting of an S-2,2,2-trichloroethoxycarbonyl derivative, an S-benzyloxycarbonyl derivative, an S-benzyl thioether derivative, an S-triphenylmethyl thioether derivative, an S-2,4,6-trimethoxybenzyl thioether derivative, and an S-2-picolyl N-oxide thioether derivative.

66. The method of claim 54 wherein said second reactive group is masked with a photosensitive masking group and said light source is ultraviolet or laser light.

67. The method of claim 54 wherein said second reactive group is masked with a photosensitive masking group having the formula: 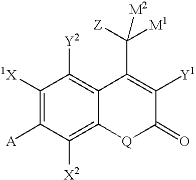
wherein,
A is —OH, substituted or unsubstituted alkoxy, —OC(O)CH₃, —NH₂, or —NHCH₃;
each of X¹ and X², independently, is H, Cl, Br, or I, at least one of X¹ and X² being Cl, Br, or I;
Q is —O—, —NH—, or —NCH₃—;
Y¹ is —H, —Cl, —Br, —I, —C(O)OH, —NO₂, —C(O)NHR¹, —CN, —C(O)H, —C(O)CH₃, benzoxazol-2-yl, benzothiazol-2-yl, or benzimidazol-2-yl;
Y² is —H, —C(O)OH, or —SO₃H; M¹ is —H, —CH₃, —NR²R³, —C(O)NR²R³, or —COOH;
Z is said second reactive group;
M² is —H, or Z and M² together are ═N₂, ═O, or ═NNHR¹; and
each of R¹, R², and R³, independently, is a substituted or unsubstituted moiety selected from the group consisting of a C₁₋₂₀ alkyl, a C₂₋₂₀ alkenyl, a C₂₋₂₀ alkynyl, a C₁₋₂₀ alkoxy, a C₁₋₂₀ thioalkoxy, a C₁₋₂₀ alkylsulfonyl, a C₄₋₁₆ arylsulfonyl, a C₂₋₂₀ heteroalkyl, a C₂₋₂₀ heteroalkenyl, a C₃₋₈ cycloalkyl, a C₃₋₈ cycloalkenyl, a C₄₋₁₆ aryl, a C₄₋₁₆ heteroaryl, and a C₂₋₃₀ heterocyclyl.

68. The method of claim 54 wherein said second reactive group is masked with a chemically sensitive masking group and said second reactive group and said chemically sensitive masking group together form a moiety selected from the group consisting of an s-alkyl thioether having the formula CₙH₂ₙ₊₁SR, an s-benzyl thioether having the formula RSCh₂Ph, and an s-diphenylmethyl thioether having the formula RSCH(C₆H₅)₂, wherein
R is said macromolecule; and
said exposing step (b) cleaves said moiety thereby unmasking said second reactive group.

69. The method of claim 54, further comprising
(c) testing for background conductance between said first electrode and said second electrode;
(d) exposing said electrode pair to a solution that potentially comprises an analyte for a period of time;
(e) drying said electrode pair; and
(f) measuring a current through said electrode pair.

70. The method of claim 69 wherein said period of time is less than 30 minutes.

71. The method of claim 69 wherein said period of time is between one minute and one hour.

72. The method of claim 69 wherein said period of time is between one hour and fifteen hours.

73. The method of claim 69 wherein said macromolecule is a single stranded nucleic acid and said analyte is a single stranded nucleic acid that has sufficient complementarity to said macromolecule to bind to said macromolecule in said exposing step.

74. The method of claim 69 wherein said macromolecule is a single stranded nucleic acid and said analyte is a single stranded nucleic acid that has sufficient complementarity to said macromolecule to bind to said macromolecule under conditions of high stringency.

75. The method of claim 69 wherein said macromolecule is a single stranded nucleic acid and said analyte is a single stranded nucleic acid that has sufficient complementarity to said macromolecule to bind to said macromolecule under conditions of intermediate stringency.

76. The method of claim 69 wherein said macromolecule is a single stranded nucleic acid and said analyte is a single stranded nucleic acid that has sufficient complementarity to said macromolecule to bind to said macromolecule under conditions of low stringency.

77. The method of claim 69 wherein said drying step (e) comprises blowing nitrogen or argon gas on said electrode pair.

78. The method of claim 69 wherein said measuring step (f) comprises quantifying a current across said electrode pair when a voltage of between ±5 volts is applied across said electrode pair.

79. The method of claim 69 wherein said analyte comprises a whole cell, a subcellular particle, a virus, a prion, a viroid, a nucleic acid, a protein, an antigen, a lipoprotein, a lipopolysaccharide, a lipid, a glycoproteins, a carbohydrate moiety, a cellulose derivative, an antibody, a fragment of an antibody, a peptide, a hormone, a pharmacological agent, a cellular component, an organic compound, a non-biological polymer, a synthetic organic molecule, an organo-metallic compound, or an inorganic molecule.

80. The method of claim 54 wherein said applying step (a) and said exposing step (b) are repeated with a different electrode pair in said plurality of electrode pairs using a different macromolecule, wherein
(i) said different electrode pair comprises a first electrode and a second electrode;
(ii) a first portion of said different macromolecule is derivatized with a first reactive group and a second portion of said macromolecule is derivatized with a second reactive group; and
(iii) said first reactive group is masked with an electrolabile masking group and said second reactive group is masked with a photosensitive or chemically sensitive masking group.

81. The method of claim 54 wherein said applying step (a) is repeated with a different electrode pair in said plurality of electrode pairs using a different macromolecule prior to said exposing step (b), wherein
(i) said different electrode pair comprises a first electrode and a second electrode;
(ii) a first portion of said different macromolecule is derivatized with a first reactive group and a second portion of said macromolecule is derivatized with said second reactive group; and
(iii) said first reactive group is masked with an electrolabile masking group and said second reactive group is masked with said photosensitive or chemically sensitive masking group.
